(19) United States
(12) Patent Application Publication
(10) Pub. No.: US 2011/0098783 A1
(43) Pub. Date: Apr. 28, 2011

(54) METHOD AND SYSTEM TO REGULATE ARMS, LEGS, HANDS AND OTHER SKELETAL MUSCLES BY NEURO-ELECTRICAL CODED SIGNALS

(76) Inventor: Eleanor Schuler, Albuquerque, NM (US)

(21) Appl. No.: 12/903,404

(22) Filed: Oct. 13, 2010

Related U.S. Application Data
(60) Provisional application No. 61/251,567, filed on Oct. 14, 2009.

(51) Int. Cl. A61N 1/36

(52) U.S. Cl. 607/48

(57) ABSTRACT

A method and system to control skeletal muscles generally comprising generating at least one coded waveform signal that is substantially similar to at least one coded waveform signal that is generated in the body and is operative in the control of at least a first skeletal muscle, and transmitting the generated waveform signal to a subject requiring medical treatment to control its first skeletal muscle. Additionally, neuro-electrical coded signals are utilized to control skeletal shoulder, arm, hand and finger muscles. Such signals may also be employed for controlling the movement and locomotion of lower limbs by means of neuro-electrical coded signals.

### CROSS-REFERENCE TO PROVISIONAL APPLICATION

This non-provisional patent application claims the benefit under 35 U.S.C. §119(e) of U.S. Provisional Patent Application Ser. No. 61/251,567 filed on Oct. 14, 2009, entitled “Method and System to Regulate Arms, Legs, Hands and Other Skeletal Muscles by Neuro-Electrical Coded Signals,” which is hereby incorporated by reference in its entirety.

### TECHNICAL FIELD

Embodiments relate generally to medical methods and systems for monitoring and controlling skeletal muscles. Additionally, embodiments relate to the control of skeletal muscles utilizing transmitted electrical nerve coded signals. Embodiments are also related to the use of neuro-electrical coded signals to control skeletal shoulder, arm, hand, and finger muscles. Embodiments are additionally related to methods and systems for controlling the movement and locomotion of lower limbs by means of neuro-electrical coded signals.

### BACKGROUND

As is well known in the art, the brain modulates (or controls) skeletal muscles via electrical signals (i.e., action potentials or waveform signals), which are transmitted through the nervous system. The nervous system includes the central nervous system, which comprises the brain and the spinal cord, and the cranial, and peripheral nervous systems, which generally comprise groups of nerve cells (i.e., neurons) and peripheral nerves that lie outside the brain and spinal cord. The various nerve networks and systems are anatomically separate, but functionally interconnected.

As indicated, the peripheral nervous system is constructed of nerve cells (or neurons) and glial cells (or glia), which support the neurons. Operative neuron units that carry signals from the brain are referred to as “efferent” nerves. “Afferent” nerves are those that carry sensor or status information to the brain. Together, these components of the nervous system are responsible for the function, regulation, and modulation of the body's organs, muscles, secretory glands, and other physiological systems.

As is known in the art, a typical neuron includes four morphologically defined regions: (i) cell body, (ii) dendrites, (iii) axon, and (iv) pre-synaptic terminals. The cell body (soma) is the metabolic center of the cell. The cell body contains the nucleus, which stores the genes of the cell, and the rough and smooth endoplasmic reticulum, which synthesizes the proteins of the cell, along with other cellular components.

The nerve cell body typically includes two types of outgrowths (or processes): the dendrites and the axon. Most neurons have several dendrites; these branch out in tree-like fashion and serve as an important apparatus for receiving and sending signals to and from other nerve cells.

The axon is the main conducting unit of the neuron. The axon carries coded electrical signals to the body's organs, skeletal muscles, and other physiological systems to control the function thereof. The axon is capable of conveying electrical signals along distances that range from as short as 0.1 mm to as long as 2 m.

Near the end of the axon, the axon may be divided into fine branches that make contact with other neurons. The point of contact is referred to as a synapse. The cell transmitting a signal is called the pre-synaptic cell. The cell receiving the signal is referred to as the postsynaptic cell. Specialized swellings on the axon's branches (i.e., pre-synaptic terminals) serve as the transmitting site in the pre-synaptic cell.

Most axons terminate near a postsynaptic neuron's dendrites. However, communication can also occur at the cell body or, less often, at the initial segment or terminal portion of the axon of the postsynaptic cell.

The electrical signals transmitted along the axon, referred to as action potentials, are rapid and transient “all-or-none” nerve impulses. Action potentials typically have an amplitude of less than approximately 100 millivolts (mV) and a duration of approximately 1 msec. Action potentials are conducted along the axon, without failure or distortion, at rates in the range of approximately 1-100 meters/sec. The amplitude of the action potential remains constant throughout the axon, since the impulse is continually regenerated as it traverses the axon by a sort of relay between like neurons (i.e., neurons firing like signals) which are a piece of the neuro signal.

As is known in the art, a “neurosignal” is a composite signal that includes many action potentials. The neurosignal also includes an instruction set for proper organ function and/or system. A skeletal muscle neurosignal would thus include an instruction set for a muscle to perform a desired movement, including information regarding initial muscle tension, degree of muscle movement, and range of muscle strength, etc.

Neurosignals or “neuro-electrical coded signals” are thus codes that contain complete sets of information for complete organ or muscle function. As set forth in U.S. Patent Application Publication No. 20050251061, entitled “Method and system to record, store and transmit waveform signals to regulate body organ function,” and published on Nov. 10, 2005, once these neurosignals, which are embodied in the “waveform signals” referred to herein, have been isolated, recorded, standardized, and transmitted to a subject (or patient), a generated nerve-specific waveform instruction (i.e., waveform signal(s)) can be employed to control a skeletal muscle and, hence, treat a multitude of muscle impairments. The noted impairments include, but are not limited to, spinal injuries, brain tumor, multiple sclerosis, cerebral palsy, radiation-induced nerve damage, stroke induced neuron damage, damage caused by explosive injury or destruction of limbs, and so forth.

As is known in the art, the contraction and movement of skeletal muscles is commanded and coordinated by a number of the aforementioned brain structures, including the cerebral cortex, cerebellum, and brain system structures. To accomplish various brain designated tasks, neurosignals are transmitted to a target skeletal muscle or muscles to induce graduated coarse or fine motor movements.

Various apparatus, systems, and methods have been developed, which include an apparatus for or step of recording action potentials or coded electrical neurosignals, to control various physiological systems. The signals are, however, typically subjected to extensive processing and are subsequently employed to operate and/or regulate a “mechanical” device or system, such as a muscle stimulator device. Illustrative are the systems disclosed in U.S. Pat. Nos. 6,360,740 and 6,651,652

In U.S. Pat. No. 6,360,740, a system and method for providing respiratory assistance is disclosed. The noted method includes the step of recording “breathing signals”, which are generated in the respiratory center of a patient. The “breathing signals” are processed and employed to control a muscle stimulation apparatus or ventilator.

In U.S. Pat. No. 5,167,229, a method and system for inducing skeletal muscle movement is disclosed. The method and system of U.S. Pat. No. 5,167,229 includes the step of implanting a sensor, i.e., input command means, in the body that is adapted to sense physical movement and provide a signal “which is indicative of a selected physiological movement or group of movements.” The signal is then processed and employed to control implanted electrodes that are adapted to stimulate target muscles.

A major drawback associated with the systems and methods disclosed in the noted prior art patents, however, as well as most known systems, is that the control signals that are generated and transmitted are “user determined” and “device determinative”. The noted “control signals” are thus not related to or representative of the signals that are generated in the body and, hence, would not be operative in the control of the skeletal muscles if transmitted thereto.

It would thus be desirable to provide a method and system for controlling skeletal muscles that includes means for generating and transmitting coded electrical neurosignals (referred to herein as “waveform signals”) to the body that substantially correspond to the recorded waveform signals and are operative naturally in the control of the skeletal muscles by the brain.

Additionally, if the medical treatment field could control muscles in the thoracic limb(s) by the actual brain generated neuro-coded signals (also “waveform signals”), there would be a potential treatment for paralyzed humans and animals that have no such treatment, presently. There are millions of people in the United States and in foreign countries who suffer from the loss or partial loss of arm and hand motion. Most of these people have suffered spinal injuries whereby there is a loss of neuro-code transmission to the important muscle groups that operate both the arms and hands. Such spinal injuries occur when the nerves are crushed, severed, or because of stroke; there is a loss of neuro-coded communication from the brain to the arm. In most spinal injury cases, for example, the arm and hand are intact, but unable to receive the neuro-coded signals that allow the brain to operate the limb. In stroke cases, one-side loss of an arm and hand is the consequence of controlling neurons in the brain having lost blood supply and have been damaged to the point that neuro-coded signals cannot be transmitted to the arm and perhaps other systems of the body on the affected side. Injuries from industrial and auto accidents as well as battlefield injuries incurred by military solders and the like can result in a variety of handicaps related to the loss of nerve control related to one or both arms and hands.

It would be beneficial medical treatment if the natural neuro-coded signals could be broadcast into the large and small nerves leading to the muscles of the thoracic limb. A computerized treatment device would be desired to return some or most arm and hand movements in paralyzed patients. Thus, a limb could receive its operating instructions in a manner equivalent to the original natural method they were born with. Present-day tactics using the nerves as a cable and sending high-voltage along the nerve to a muscle does not necessarily cause smooth, accurate, and useful control. Nor has any present-day high-voltage method found commercial acceptance by quadriplegic and paraplegic patients. Injured soldiers who are usually young require more elegant control of limbs, hands, fingers, and so forth.

All of the skeletal muscles of the shoulder, arm, forearm, wrist, and fingers of humans or animals are operated by the brain via nerve signals. Muscle contraction and movement is coordinated and commanded by a number of brain structures including the cerebral cortex, cerebellum, and brainstem structures. Instructions in the form of neuro-electrical signals travel within the nerves to the muscles. Such signals cause graduated coarse or fine motor movements to accomplish the various brain designated tasks. The speed of movement is also controlled by the brain's neuro signals.

### BRIEF SUMMARY

The following summary is provided to facilitate an understanding of some of the innovative features unique to the disclosed embodiments and is not intended to be a full description. A full appreciation of the various aspects of the embodiments disclosed herein can be gained by taking the entire specification, claims, drawings, and abstract as a whole.

It is therefore an object of the disclosed embodiments to provide a method and system for controlling the arms, hands, fingers, and other skeletal muscles that overcomes the drawbacks associated with prior art methods and systems for controlling skeletal muscles.

It is another object of the disclosed embodiments to provide a method and system for controlling skeletal muscles that includes means for generating skeletal muscle waveform signals that substantially correspond to coded waveform signals that are generated in the body and are operative in the control of skeletal muscles.

It is another object of the disclosed embodiments to provide a method and system for controlling skeletal muscles that includes means for recording waveform signals that are generated in the body and operative in the control of skeletal muscles.

It is another object of the disclosed embodiments to provide a method and system for controlling skeletal muscles that includes processing means adapted to generate at least one base-line skeletal muscle signal that is representative of at least one coded waveform signal generated in the body from recorded waveform signals and thus exhibit a normal range of operation.

It is another object of the disclosed embodiments to provide a method and system for controlling skeletal muscles that includes processing means adapted to compare recorded skeletal muscle waveform signals to baseline skeletal muscle signals and generate a skeletal muscle signal based on the comparison of the signals.

It is another object of the disclosed embodiments to provide a method and system for controlling skeletal muscles that includes monitoring means for detecting skeletal muscle impairments and disorders.

It is another object of the disclosed embodiments to provide a method and system for controlling skeletal muscles that includes means for transmitting waveform signals to the body that substantially correspond to coded waveform signals that are generated in the body and are operative in the control of the skeletal muscles.

It is another object of the disclosed embodiments to provide a method and system for controlling skeletal muscles that includes means for transmitting signals directly to the nervous system in the body that substantially correspond to coded waveform signals that are generated in the body and are operative in the control of the skeletal muscles.

It is another object of the disclosed embodiments to provide a method and system for controlling skeletal muscles that can be readily employed in the treatment of muscle and nerve related disorders and abnormalities, including spinal injuries and muscle nerve damage.

In accordance with the above objects and those that will be mentioned and will become apparent below, the method to control skeletal muscles in one embodiment generally comprises (i) generating at least a first coded waveform signal that substantially corresponds to at least one coded waveform signal that is generated in the body and is recognizable by at least a first skeletal muscle as a control signal and (ii) transmitting the first waveform signal to a subject to control the first skeletal muscle.

In another embodiment, the method to control skeletal muscles generally comprises (i) capturing coded waveform signals that are generated in the body and are operative in the control of at least a first skeletal muscle, (ii) generating at least a first waveform signal that is recognizable by the first skeletal muscle as a control signal, and (iii) transmitting the first waveform signal to a subject to control the first skeletal muscle.

In one embodiment, the first waveform signal includes at least a second waveform signal that substantially corresponds to at least one of the captured waveform signals and is operative in the control of the first skeletal muscle.

In one embodiment, the first waveform signal is transmitted to the subject's nervous system. In another embodiment, the first waveform signal is transmitted proximate to a target zone on the neck, head, or spinal region.

In another embodiment, the method to control skeletal muscles generally comprises (i) capturing coded waveform signals that are generated in the body and are operative in the control of skeletal muscles, (ii) storing the captured waveform signals in a storage medium, the storage medium being adapted to store the components of the captured waveform signals according to the function performed by the waveform signal components, (iii) generating at least a first waveform signal that substantially corresponds to at least one of the captured waveform signals and is operative in the control of at least a first skeletal muscle, and (iv) transmitting the first waveform signal to an injured subject requiring functional use of a limb, for example, to control the first skeletal muscle.

In another embodiment, the method to control skeletal muscles generally comprises (i) capturing a plurality of waveform signals generated in a first subject's body that are operative in the control of skeletal muscles, (ii) generating a base-line skeletal muscle waveform signal from the plurality of captured waveform signals, the base-line skeletal muscle waveform being operative in the control of a first skeletal muscle, (iii) capturing a second waveform signal generated in the first subject's body that is operative in the control of the first skeletal muscle, (iv) comparing the base-line waveform signal to the second waveform signal, (v) generating a third waveform signal based on the comparison of the base-line and second waveform signals, and (vi) transmitting the third waveform signal to the first subject's body, the third waveform signal being operative in the control of the first skeletal muscle.

In one embodiment, the plurality of waveform signals is captured from a second subject's body that has no malfunction of limb neurocodes. Such normal signals are transferred to storage media for future use as a treatment signal.

In another embodiment, the plurality of waveform signals is captured from a plurality of subjects to be incorporated into a treatment library installed in, for example, a hybrid scientific computer.

Preferably, the third waveform signal is transmitted to the subject's nervous system. In an alternative embodiment, the third waveform signal is transmitted proximate to a target zone on the neck, head, or spinal region.

In accordance with a further embodiment, the method for controlling skeletal muscles generally comprises (i) monitoring the status of at least a first skeletal muscle of a subject, (ii) providing at least one skeletal muscle status signal in response to a skeletal muscle disorder of the first skeletal muscle, (iii) generating at least a first waveform signal that is operative in the control of the first skeletal muscle in response to the skeletal muscle status signal, and (iv) transmitting the first waveform signal to the subject to mitigate the skeletal muscle disorder.

In accordance with a further embodiment, the method for controlling skeletal muscles generally comprises (i) capturing waveform signals that are generated in the body and are operative in control of skeletal muscles, the waveform signals including at least a first waveform signal that is operative in the control of a first skeletal muscle, (ii) monitoring the skeletal muscle status of the first skeletal muscle of a subject and providing at least one skeletal muscle status signal indicative of the status of the first skeletal muscle, (iii) storing the captured waveform signals and skeletal muscle status signal in a storage medium, (iv) generating at least a first waveform that is operative in the control of the first skeletal muscle in response to a skeletal muscle status signal or component of a captured waveform signal that is indicative of a skeletal muscle disorder, and (v) transmitting the first waveform signal to the subject to mitigate the skeletal muscle disorder.

In yet another embodiment, the method to control skeletal muscles generally comprises (i) capturing a first plurality of waveform signals generated in the body that are operative in the control of skeletal muscles, (ii) capturing at least a first waveform signal from a subject's body that is indicative of a skeletal muscle disorder, (iii) generating a confounding signal that is operative to mitigate the skeletal muscle disorder, and (iv) transmitting the confounding waveform signal to the subject to mitigate the skeletal muscle disorder.

The system to control skeletal muscles, in accordance with one embodiment, generally comprises (i) at least a first signal probe adapted to capture coded waveform signals from the body, the waveform signals being representative of waveform signals naturally generated in the body and operative in the control of skeletal muscles, (ii) a processor in communication with the signal probe and adapted to receive the waveform signals, the processor being further adapted to generate at least a first waveform signal based on the captured waveform signals, the first waveform signal being recognizable by at least a first skeletal muscle as a control signal, and (iii) at least a second signal probe adapted to be in communication with a subject's body for transmitting the first waveform signal to the body to control the first skeletal muscle.

Preferably, the processor includes a storage medium adapted to store the captured waveform signals.

In one embodiment, the processor can be adapted to extract and store components of the captured waveform signals in the storage means according to the function performed by the signal components.

In another embodiment, a method and system is provided for turning on the neuro-electrical signals that cause controlled major and minor movements in the muscles of the lower limbs. Such an approach may be utilized for medical treatment use in individual animals or humans who have lost the use of a muscle or groups of muscles (e.g., as a result of military action) or who have suffered a spinal injury or a disease that prevents them from locomoting such as, for example, MS (Multiple Sclerosis).

Neuro electric coded signals may be captured from a plurality of mammals for all of the mammalian muscles from the waist downward that are involved in standing, leaning, walking, running, or balancing in the up right or other positions. The signals can be used as medical treatment in a plurality of humans or animals that require activation of locomotion and balancing muscles to improve their life and health. The captured signals can be stored in a computer memory or other electronic memory component and then adjusted by a data-processing device or system for application to each muscle that is impaired. An orchestrating computer can be utilized to organize the individual codes so as to perform the locomotion or standing or balancing movement. Individual electrodes or transmitting devices are hooked or brought into close proximity to each muscle that is to be activated.

As a practical matter it can be expected that an additional voltage up to 5 volts may be required to allow signal transport through any natural nerve insulation or resistance at the connection before it reaches the neurons that originate and relay the signal for actual leg muscle movement and to include walking, running, kicking, and for lifting heavy objects.

The disclosed embodiments utilize a neurological signal scientific computer system to collect and store the actual coded signals produced by mammalian brains and brainstem. Such stored signals can then be broadcast onto or into the Sciatic nerve and its branches to activate and regulate the muscles of the leg. The amount of movement or locomotion and the frequency of movement are controllable with variations in the neuro-coded signals.

The disclosed embodiments provide a system and method to broadcast the leg motion neuron-codes previously collected from a mammal's respiratory tract. Such neuro-codes are the neuron generated codes that travel from the brain and brainstem onto and within the nerves of the leg. Neuro-coded signals coming from the brain and brainstem are the effector signals (efferent) and the signals coming from the Sciatic nerve travel to the brain are the afferent or sensory nerves which report on the status of leg muscle movement. The activation and operation of the leg function fully depends on the signals traveling on and within the Sciatic and associated nerves.

### BRIEF DESCRIPTION OF THE DRAWINGS

The accompanying figures, in which like reference numerals refer to identical or functionally-similar elements throughout the separate views and which are incorporated in and form a part of the specification, further illustrate the present disclosed embodiments and, together with the detailed description herein, serve to explain the principles of the disclosed embodiments.

FIGS. 1A through 1D are illustrations of waveform signals captured from the body that are operative in the control of the skeletal muscles of the arm, forearm, hands and fingers;

FIG. 2 is an illustration of the skeletal muscles of the upper body (posterior view);

FIG. 3 is an illustration of the skeletal muscles of the right shoulder and chest regions (anterior view);

FIG. 4 is an illustration of the skeletal muscles of the right arm (anterior view);

FIG. 5 is a further illustration of the skeletal muscles of the right arm, showing the deep layer muscle structure (anterior view);

FIG. 6 is an illustration of the skeletal muscles of the right arm (posterior view);

FIG. 7 is a further illustration of the skeletal muscles of the right arm, showing the deep layer muscle structure (posterior view);

FIGS. 8A and 8B are illustrations of the skeletal muscles of the right forearm (posterior views);

FIG. 9 is an illustration of the skeletal muscles of the right hand (anterior view);

FIG. 10 is a schematic illustration of one embodiment of a skeletal muscle control system;

FIG. 11 is a schematic illustration of another embodiment of a skeletal muscle control system;

FIG. 12 is a schematic illustration of another embodiment of a skeletal muscle control system;

FIG. 13 is a schematic illustration of yet another embodiment of a skeletal muscle control system;

FIG. 14 illustrates a diagram of a neuro-electrical coded signal that operates the radial nerve in the arm, forearm, hands and fingers, in accordance with the disclosed embodiment;

FIG. 15 illustrates a diagram of a neuro-electrical coded signal that raises the arm with the hand pulling backward and the fingers spread in a medium range of movement, in accordance with the disclosed embodiments;

FIG. 16 illustrates a diagram of a neuro-electrical coded signal that produces smaller and finer forearm movement including lifting the arm up and twitching the hand backwards and to provide minor finger spreading, in accordance with the disclosed embodiment;

FIG. 17 illustrates a diagram of a neuro-electrical coded signal that provides large power movements in muscles served by the ulnar nerve, in accordance with the disclosed embodiments;

FIG. 18 illustrates a diagram of a neuro-electrical coded signal that demonstrates and causes movement via the ulnar nerve of the forearm movement downward with wrist flicks downward and minor finger movements, in accordance with the disclosed embodiments;

FIG. 19 illustrates a diagram of a neuro-electrical coded signal charged with making smaller movements via the ulnar nerve to appropriate muscles of the forearm downward with smaller wrist twitches also in a downward direction, in accordance with the disclosed embodiments;

FIG. 20 illustrates a diagram of a neuro-electrical coded signal indicative of the supra scapular nerve providing major movement of the arm by pulling upward and somewhat outward;

FIG. 21 illustrates a diagram of a neuro-electrical coded signal indicative of the supra scapular nerve to activate moderate movement of the arm by pulling it in an upward and outward direction, in accordance with the disclosed embodiments; and

FIG. 22 illustrates a diagram of a neuro-electrical coded signal carried by the scapular nerve to move the arm and shoulder to work powerfully in a downward digging or pawing the ground motion, in accordance with the disclosed embodiments.

### DETAILED DESCRIPTION

The particular values and configurations discussed in these non-limiting examples can be varied and are cited merely to illustrate at least one embodiment and are not intended to limit the scope thereof.

Before describing the disclosed embodiments including alternative embodiments in detail, it is to be understood that such embodiments are not limited to particularly exemplified apparatus, systems, structures or methods as such may, of course, vary. Thus, although a number of apparatus, systems, and methods similar or equivalent to those described herein can be used in the practice of such embodiments, the preferred systems and methods are described herein.

It is also to be understood that the terminology used herein is for the purpose of describing particular embodiments only and is not intended to be limiting.

Unless defined otherwise, all technical and scientific terms used herein have the same meaning as commonly understood by one having ordinary skill in the art to which the disclosed embodiments pertain.

Further, all publications, patents, and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “a waveform signal” includes two or more such signals; reference to “a skeletal muscle disorder” includes two or more such disorders and the like.

The term “nervous system”, as used herein, means and includes the central nervous system including the spinal cord, medulla oblongata, pons, cerebellum, midbrain, diencephalon, and cerebral hemispheres, and the cranial and peripheral nervous systems, including the neurons and glia. In addition, the nervous system can include motor pathways, motor cortex, motor neurons, motor endplate, descending motor pathways, and the like.

The terms “coded waveform signal” and “waveform signal”, as used herein, mean and include a composite electrical signal that is generated in the body and carried by neurons in the body including neurocodes, neurosignals and components and segments thereof. Note that the terms “neuro-coded electrical signals,” “neurosignals,” “neuro-signals,” “neuro-electrical coded signals,” “neuro-coded signals,” “neuro-code,” “neuro code” and variations thereof can be utilized interchangeably to refer to the same type of signal. In some instances, for example, a neuro-electrical or neuro-coded signal, etc., may be referred to simply as a “signal” for simplicity sake.

The term “skeletal muscle”, as used herein, means and includes a striated muscle, normally having at least one attachment to the skeletal system, whose contraction and extension are controlled or mediated by cognitive action.

The term “target zone”, as used herein, means and includes, without limitation, a region of the body proximal to a portion of the nervous system wherein the application of electrical signals can induce the desired neural control without the direct application (or conduction) of the signals to a target nerve.

The terms “patient” and “subject”, as used herein, mean and include humans and animals.

The term “plexus”, as used herein, means and includes a branching or tangle of nerve fibers outside the central nervous system.

The term “ganglion”, as used herein, means and includes a group or groups of nerve cell bodies located outside the central nervous system.

The terms “skeletal muscle impairment” and “skeletal muscle disorder”, as used herein, mean and include any dysfunction of a skeletal muscle that impedes the normal function thereof. Such dysfunction can be caused by a multitude of known factors and events including, without limitation, spinal cord injury and severance, a brain tumor, multiple sclerosis, cerebral palsy, and involuntary muscle contractions.

The term “module” as used herein may refer to a physical modular component such as, for example, a microprocessor, a computer memory, and the like. The term “module” may also refer to a data-processing apparatus such as a computing device, a scientific computer, and so forth, which include microprocessors, computer memory components, and so forth. The signals found in all skeletal muscles are analog. Therefore all treatment signals must also be in analog form.

The term “module” may also refer to a software or program module that includes computer-executable instructions capable of being executed by a computer or data-processing apparatus. Generally, program or software modules include, but are not limited to, routines, subroutines, software applications, programs, objects, components, data structures, etc., that perform particular tasks or implement particular abstract data types and instructions. Moreover, those skilled in the art will appreciate that the disclosed methods and systems may be practiced with other computer system configurations such as, for example, hand-held devices, multi-processor systems, data networks, microprocessor-based or programmable consumer electronics, networked PCs, minicomputers, mainframe computers, servers, and the like. The disclosed methods and systems can also be practiced with computer and data-processing systems and devices, including a neuro-signal storage and programming scientific computer known as a Neuriac®.

The term module as utilized herein may refer to a collection of routines and data structures that perform a particular task or implements a particular abstract data type. In such a situation, a module may be composed of two parts: an interface, which lists the constants, data types, variable, and routines that can be accessed by other modules or routines, and an implementation, which is typically private (accessible only to that module) and which includes source code that actually implements the routines in the module. The term module may also simply refer to an application such as a computer program designed to assist in the performance of a specific task.

The disclosed embodiments substantially reduce or eliminate the disadvantages and drawbacks associated with prior art methods and systems for controlling skeletal muscles. In one embodiment, the system for controlling skeletal muscles generally comprises means for generating at least one waveform signal that substantially corresponds to at least one waveform signal (i.e., coded electrical neurosignal) that is generated in the body and is operative in the control of at least a first skeletal muscle and means for transmitting the waveform signal to a subject's body. In an embodiment, the waveform signal is transmitted to the subject's nervous system.

In a further embodiment, the system includes means for recording waveform signals from a subject's body that are operative in the control of at least the first skeletal muscle. According to the disclosed embodiments, the “subject” can be the same subject that the generated waveform signals are transmitted to or a different subject.

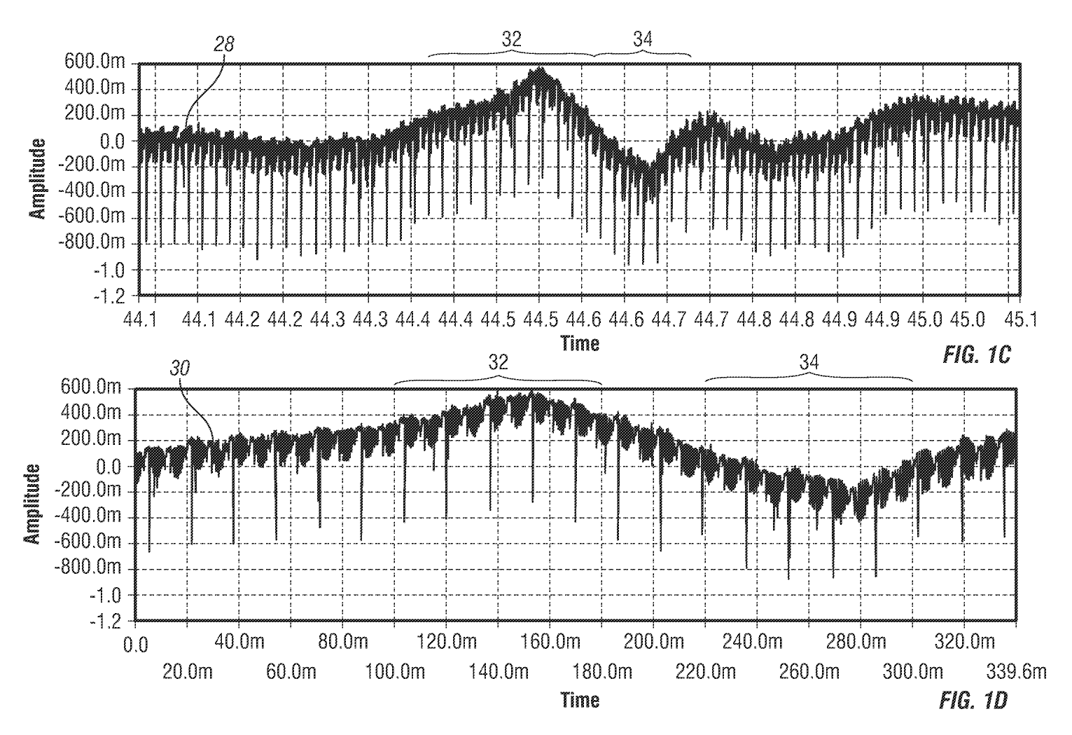

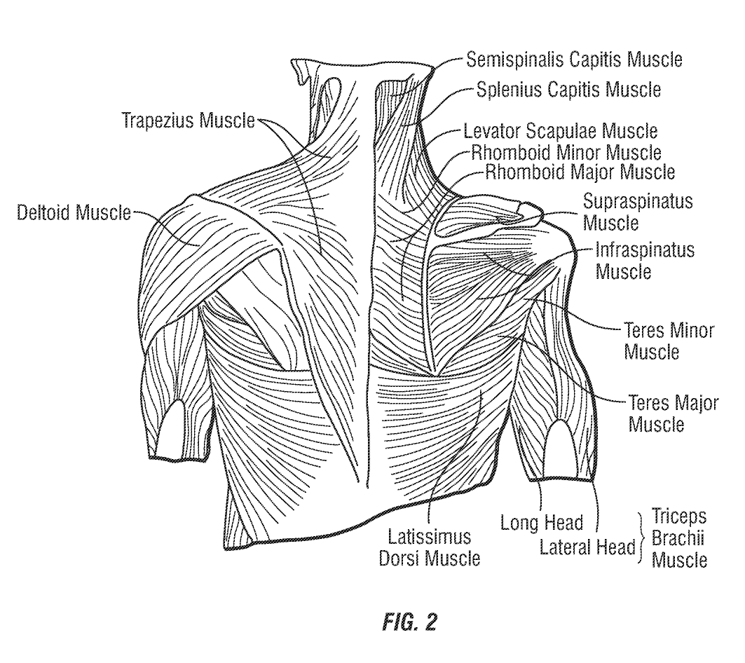

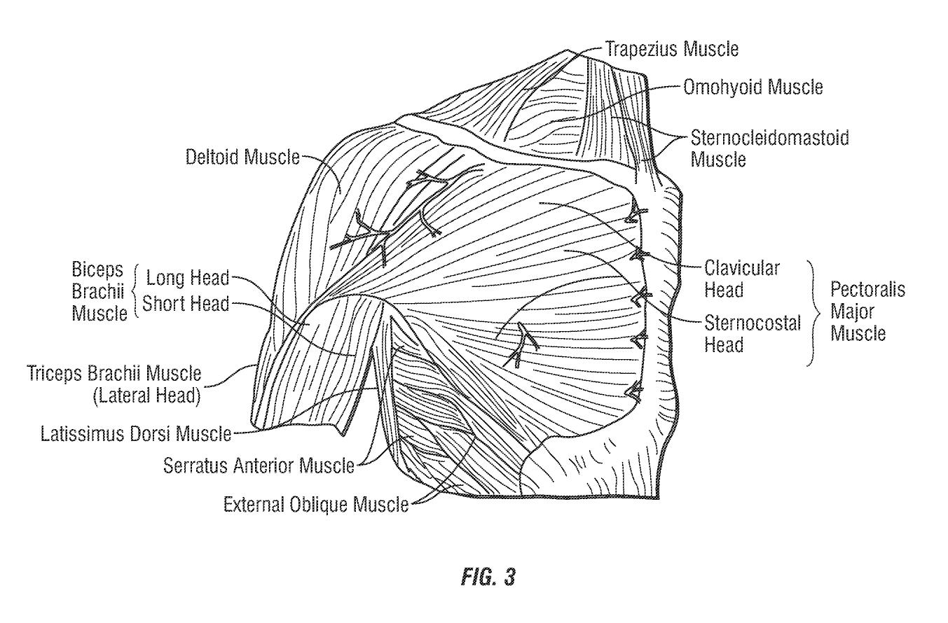

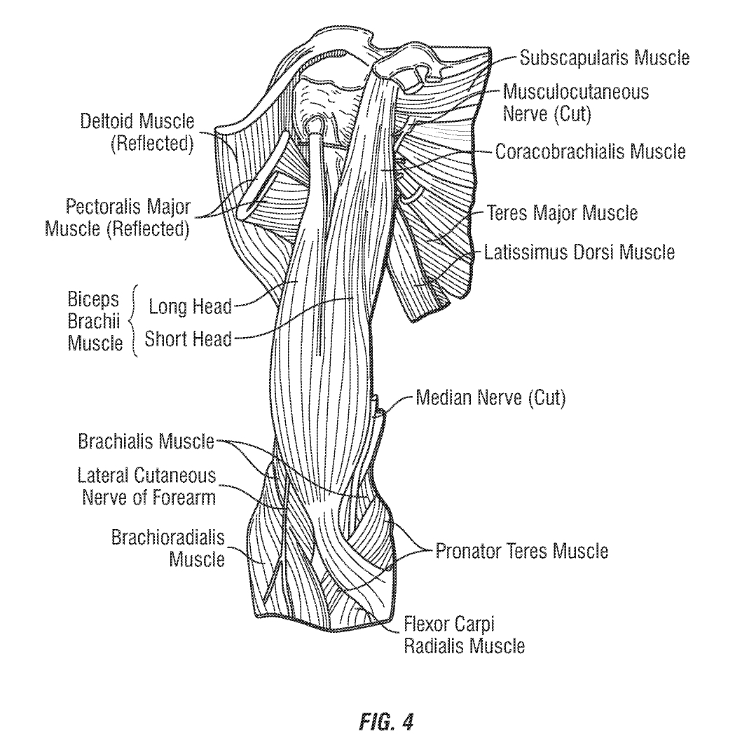

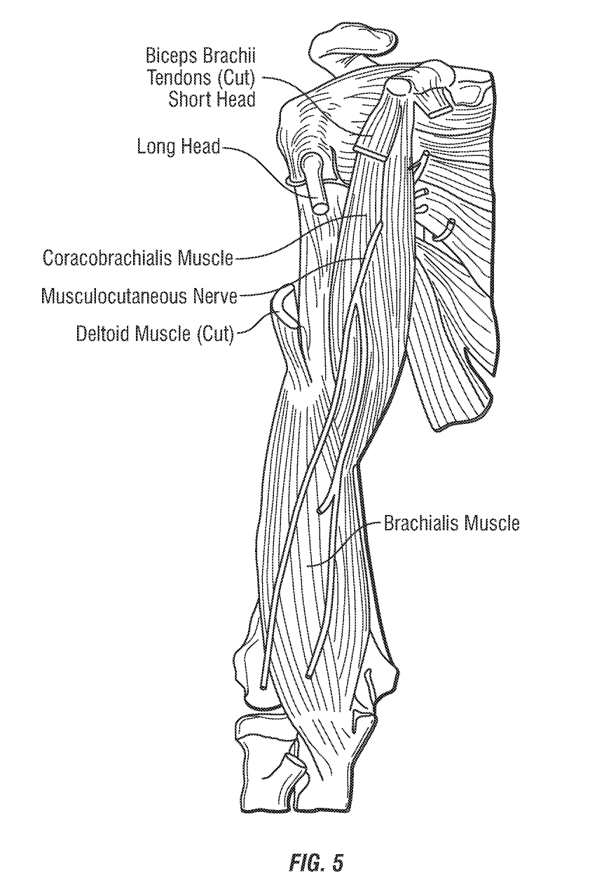

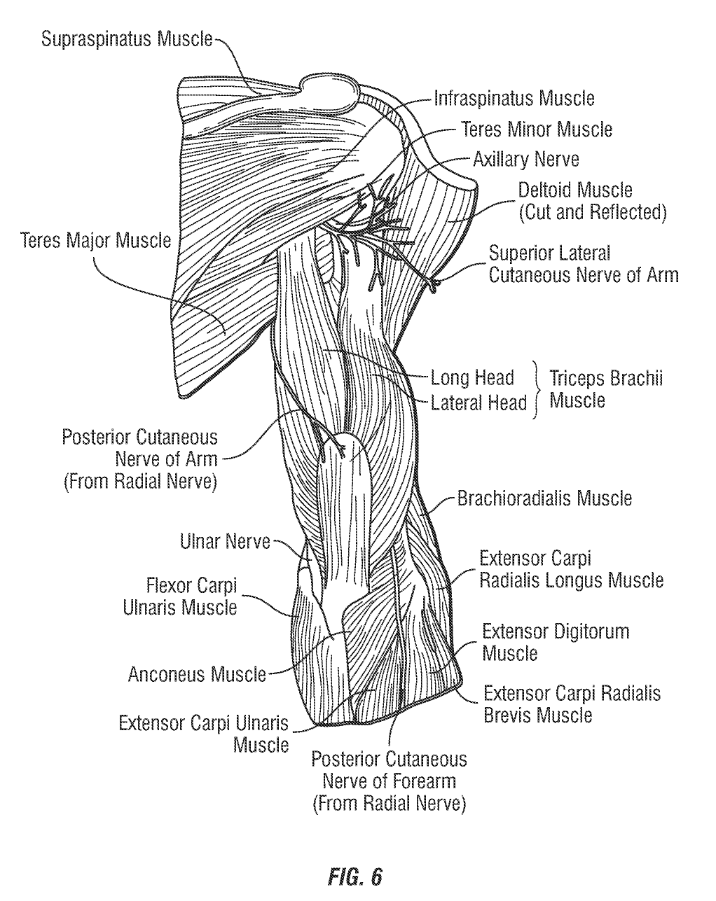

Referring now to FIGS. 2 through 9, there are shown illustrations of various skeletal muscles and muscle structures of the upper body, which can be controlled through the use of the methods and system of the disclosed embodiments. As illustrated in FIGS. 2 through 7, the skeletal muscles of the shoulder and upper arm include the levator scapulae, major and minor rhomboids, deltoids, supraspinatus, trapezius, pectoralis, coracobrachialis, biceps and triceps brachii, and latissimus dorsi.

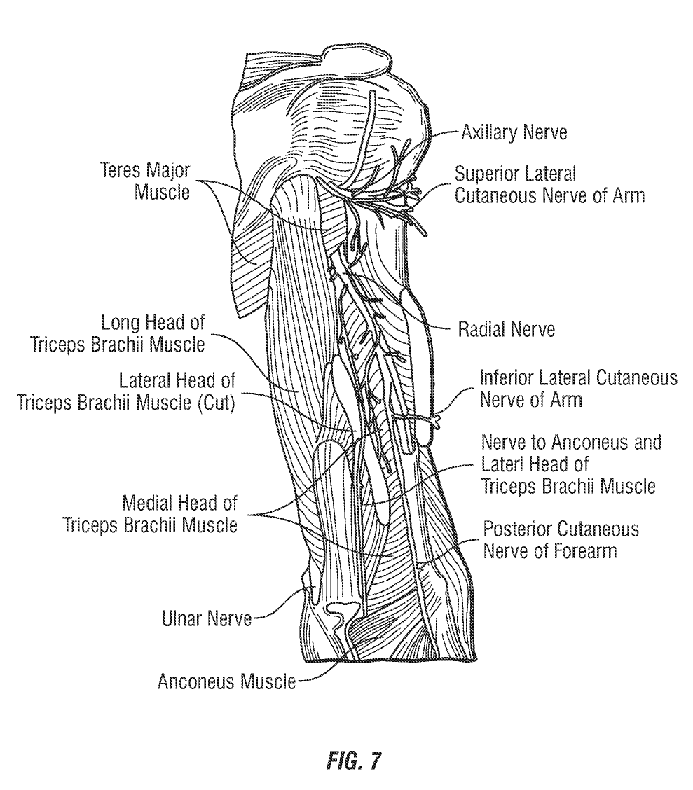

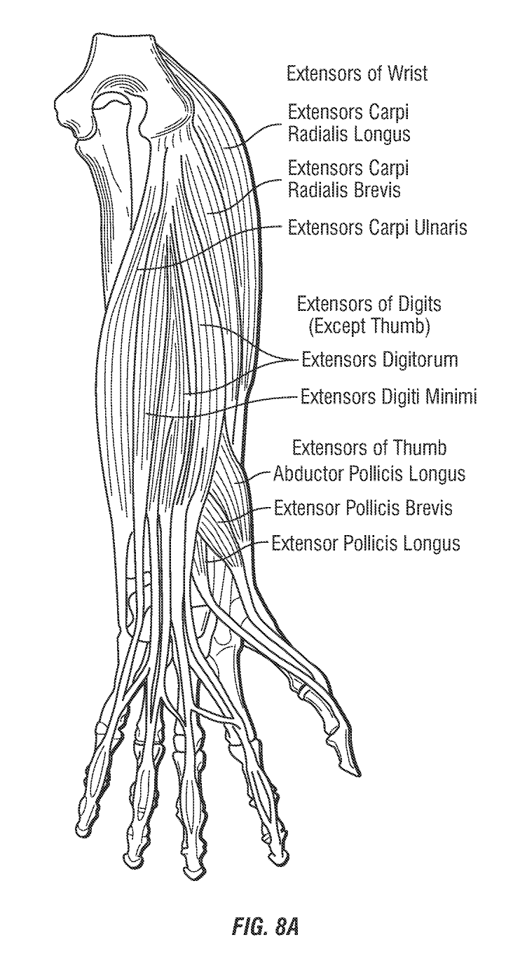

As illustrated in FIGS. 8A, 8B and 9, the skeletal muscles of the forearm, wrist, and hand include the extensor and flexor digitorums, extensor carpi ulnaris, abductor and flexor pollicis longus, lumbrical, opponens and adductor pollicis muscles, and finger and wrist flexors.

It is to be understood that, although only the skeletal muscles of the upper body are illustrated, the skeletal muscles of the lower body legs and feet are similarly within the scope of the disclosed embodiments. Such skeletal muscles include, without limitation, the quadriceps, hamstrings, adductor longus, vastus lateralis, intermedius and medialis muscles, and the sartorius.

As indicated, coded waveform signals related to skeletal muscle operation and control originate in various brain structures. The waveform signals are primarily transmitted through the spinal cord. The waveform signals that control the noted skeletal muscles of the shoulder, arm, wrist, and hand are also transmitted through the brachial plexus, and the radial, median, and ulnar nerves.

According to the disclosed embodiments, the waveform signals that control a target skeletal muscle or muscles can be captured or collected along any of the nerves carrying the waveform signals to the target skeletal muscle. By way of example, the waveform signals transmitted to the abductor pollicis muscle of the hand can be captured from the brachial plexus.

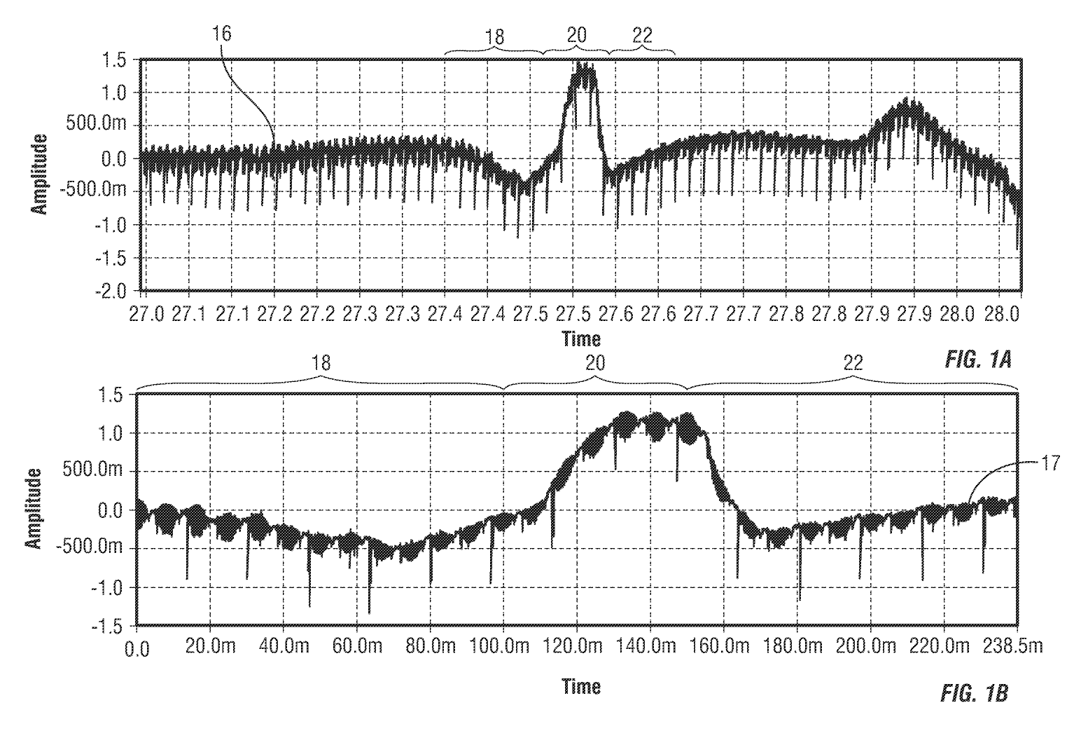

Referring now to FIGS. 1A through 1D, there are shown exemplar waveform signals that are operative in the control of the skeletal muscles of the arm, forearm, hands, and fingers. The signals 16, 17 shown in FIGS. 1A and 1B bring the arm upward and pull the hand back with the fingers spread. The signals 28, 30 shown FIGS. 1C and 1D provide the same movement as the signals shown in FIGS. 1A and 1B with less intensity (i.e., moderate movement).

As illustrated in FIGS. 1A and 1B, each signal 16, 17 includes a negative segment 18, which is believed to reflect the muscle and/or nerve setting up for movement. Following the negative segment 18 is a large positive segment 20, which produces the desired movement, and a negative segment 22, thereafter reflecting the rest and evaluation segment of the signal.

As stated above, the noted signals include coded information related to muscle movement function, such as initial muscle tension, degree (or depth) of muscle movement, etc.

In accordance with an embodiment, coded waveform signals generated in the body that are operative in the control of skeletal muscles, such as the signals shown in FIGS. 1A and 1B, can be captured and transmitted to a processor or control module. Preferably, the control module includes storage means adapted to store the captured signals. In a preferred embodiment, the control module can be further adapted to store the components of the captured signals (that are extracted by the processor) in the storage means according to the function performed by the signal components.

According to the disclosed embodiments, the stored signals can subsequently be employed to establish at least one, preferably, multiple base-line skeletal muscle waveform signals. The module can then be programmed to compare skeletal muscle waveform signals (and components thereof) captured from a subject and, as discussed below, generate at least one waveform signal or modified base-line waveform signal for transmission to the same or a different subject. Such modification can include, for example, increasing the amplitude of a skeletal muscle signal to provide a quicker or more powerful muscle movement.

According to the disclosed embodiments, the captured waveform signals can be preferably processed by proprietary means and a waveform signal (i.e., coded electrical neurosignal) that is representative of at least one captured waveform signal and operative in the control of at least one skeletal muscle (i.e., recognized by the brain or at least one skeletal muscle as a control signal) is generated by the control module. The noted waveform signal is preferably similarly stored in the storage means of the control module.

In accordance with an embodiment, the generated waveform signal is accessed from the storage means and transmitted to the subject via a transmitter (or treatment member) to control a target skeletal muscle or muscles. As discussed in detail herein, various transmitters can be employed within the scope of the embodiments to transmit the generated waveform signals to a subject.

The applied voltage of a transmitted waveform signal (or signals) can be up to 3 volts to allow for voltage loss during the transmission of the signals. Preferably, current is maintained to less than 2 amp output. Direct conduction into the nerves via electrodes connected directly to such nerves preferably have outputs less than 3 volts and current less than one tenth of an amp. The operating voltage is preferably (but not necessarily) around 1/10^{th} of a volt. Note that individual neurons can run on less than 1 volt, in micro volts and pico amps.

As discussed in detail in U.S. Patent Application Publication No. 20050251061, entitled “Method and system to record, store and transmit waveform signals to regulate body organ function,” published on Nov. 10, 2005, varying the voltage of transmitted waveform signals causes movement changes, which are generally proportional to the voltage change. For example, a waveform signal delivered at a slightly higher voltage will cause a stronger and larger muscle movement. Likewise, the same waveform signal delivered at a slightly lower voltage will cause a lesser and smaller movement of the target muscle(s). Note that U.S. Patent Application Publication No. 20050251061 is incorporated herein by reference in its entirety. Additionally, U.S. Patent Application Publication No. 20040260360, entitled “Skeletal muscle control by means of neuro-electrical signals,” which published on Dec. 23, 2004 is also incorporated herein by reference in its entirety.

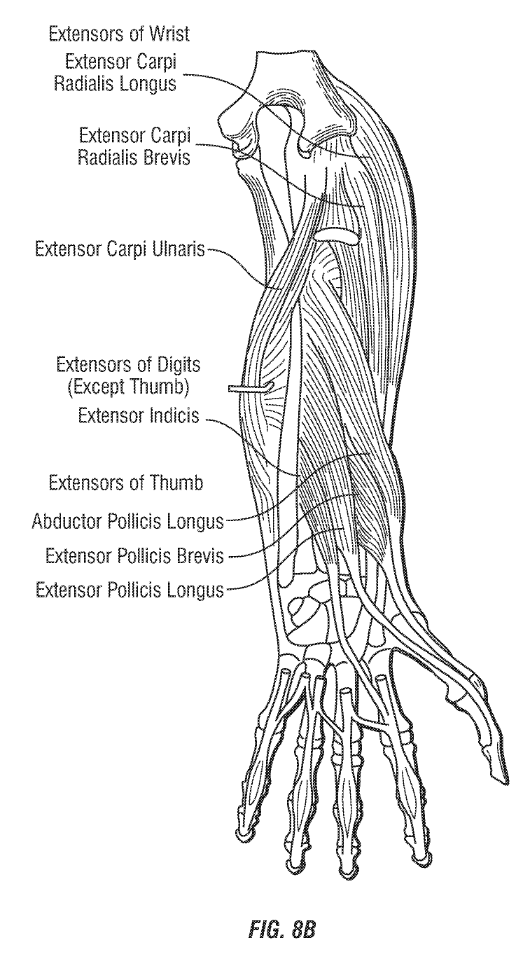

Referring now to FIG. 10, there is shown a schematic illustration of one embodiment of a skeletal muscle control system 20A. As illustrated in FIG. 10, the control system 20A includes a control module 22, which is adapted to receive coded neurosignals or “waveform signals” from a skeletal muscle signal sensor (shown in phantom and designated 21) that is in communication with a subject, and at least one treatment member 24.

The treatment member 24 can be adapted to communicate with the body and receives generated waveform signals from the control module 22. The treatment member 24 can be configured to include an electrode, antenna, a seismic transducer, or any other suitable form of conduction attachment for transmitting skeletal muscle waveform signals that control skeletal muscle function in human and animals.

The treatment member 24 can be attached to appropriate nerves via a surgical process. Such surgery can, for example, be accomplished with “key-hole” entrance in a thoracic-stereo-scope procedure. If necessary, a more expansive thoracotomy or other surgical approach can be employed for placement of the treatment member 24.

As illustrated in FIG. 10, the control module 22 and treatment member 24 can be entirely separate elements, which allow system 20A to be operated remotely. The control module 22 may be unique (e.g., tailored to a specific operation and/or subject) or may constitute a conventional device.

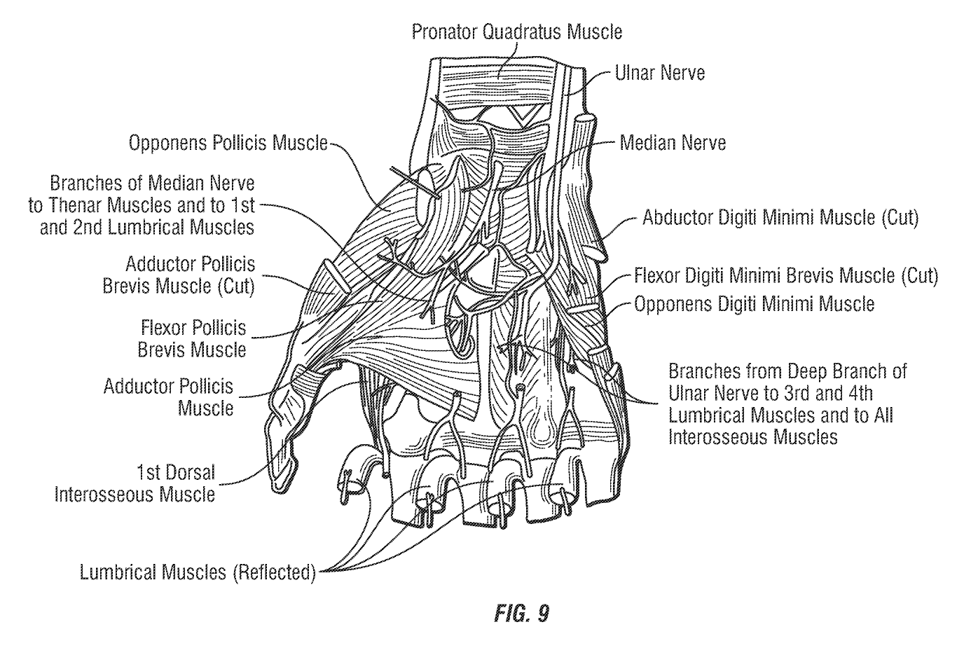

Referring now to FIG. 11, there is shown a further embodiment of a control system 20B. As illustrated in FIG. 11, the system 20B is similar to system 20A shown in FIG. 10. However, in this embodiment, the control module 22 and treatment member 24 are connected.

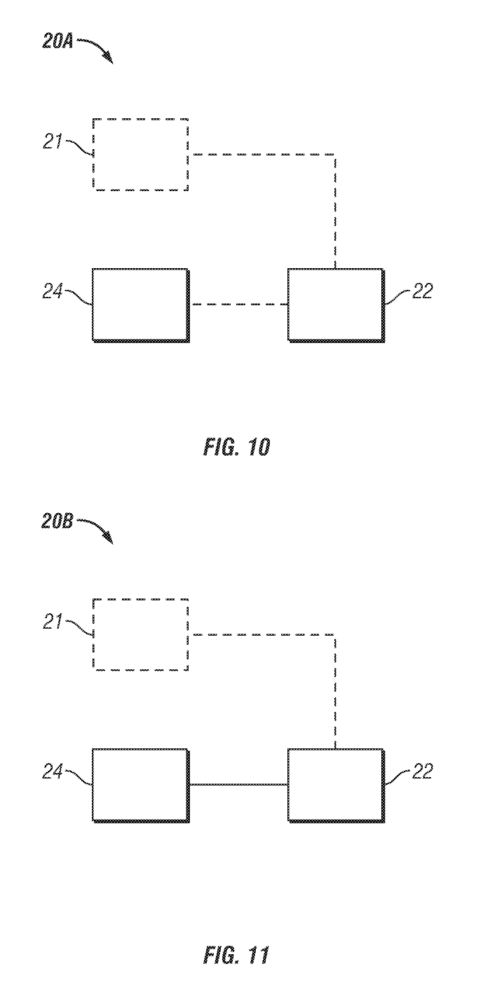

Referring now to FIG. 12, there is shown yet another embodiment of a control system 20C. As illustrated in FIG. 12, the control system 20C similarly includes a control module 22 and a treatment member 24. The system 20C further includes at least one skeletal muscle signal sensor 21.

The system 20C can also include a processing module 26 (or scientific computer). The processing module 26 can be a separate component or can be a sub-system of a control module 22′, as shown in phantom.

As indicated above, the processing module (or control module) 26 preferably includes a storage means adapted to store the captured skeletal muscle waveform signals. In a preferred embodiment, the processing module 26 is further adapted to extract and store the components of the captured skeletal muscle waveform signals in the storage means according to the function performed by the signal components.

In one embodiment, the method for controlling skeletal muscles includes the following steps: (i) generating at least a first coded waveform signal that substantially corresponds to at least one coded waveform signal that is generated in the body and is recognizable by at least a first skeletal muscle as a control signal which is stored, and (ii) transmitting the first waveform signal to an injured subject to control the first skeletal muscle.

In another embodiment, the first waveform signal is transmitted to the subject's nervous system. In another embodiment, the first waveform signal is transmitted proximate to a target zone on the neck, head, or spinal region.

According to the disclosed embodiments, the generated waveform signal is preferably transmitted to the subject via a constant current or constant voltage method.

The constant current method allows for the voltage level to fluctuate as the resistance changes. In one embodiment, a positive and negative probe (the negative probe located cranial to the positive probe) is attached to a target nerve. The distance between the probes is preferably approximately 2 cm. A ground connection is also made between the interior muscles and an earth ground.

In the constant voltage method, a signal probe is attached to the target nerve. While the signal probe is capable of providing both the positive and negative portions of the neuro-code, only the positive portion of the neuro-code is used to stimulate the nerve. The signal ground probe is not required. A ground connection is similarly made between the interior muscles and an earth ground.

In another embodiment, the method to control skeletal muscles generally comprises (i) capturing waveform signals that are generated in the body and are operative in the control of at least a first skeletal muscle, (ii) generating at least a first waveform signal that is recognizable by the first skeletal muscle as a control signal, and (iii) transmitting the first waveform signal to a subject to control the first skeletal muscle.

In a preferred embodiment, the first waveform signal includes at least a second waveform signal that substantially corresponds to at least one of the captured waveform signals and is operative in the control of the first skeletal muscle of an injured subject.

In an alternative embodiment, the first waveform signal is transmitted to the subject's nervous system. In another embodiment, the first waveform signal is transmitted proximate to a target zone on the neck, head, or spinal region and directed at specific nerves associated with muscles to be controlled and/or regulated.

In another embodiment, the method to control skeletal muscles generally comprises (i) capturing waveform signals that are generated in the body and are operative in control of skeletal muscles, (ii) storing the captured waveform signals in a storage medium, the storage medium being adapted to store the components of the captured waveform signals according to the function performed by the signal components, (iii) generating at least a first waveform signal that substantially corresponds to at least one of the captured waveform signals and is operative in the control of at least a first skeletal muscle, and (iv) transmitting the first waveform signal to a subject.

In still another embodiment, the method to control skeletal muscles generally comprises (i) capturing a first plurality of waveform signals generated in a first subject's body that are operative in the control of skeletal muscles, (ii) generating a base-line skeletal muscle waveform signal from the first plurality of waveform signals, the base-line skeletal muscle waveform being operative in the control of a first skeletal muscle, (iii) capturing a second waveform signal generated in the first subject's body that is operative in the control of the first skeletal muscle, (iv) comparing the base-line waveform signal to the second waveform signal, (v) generating a third waveform signal based on the comparison of the base-line and second waveform signals, and (vi) transmitting the third waveform signal to the first subject, the third waveform signal being operative in the control of the first or another affected skeletal muscle requiring treatment.

In one embodiment, the first plurality of waveform signals is captured from a second subject's body.

In another embodiment, the first plurality of waveform signals is captured from a plurality of subjects.

In one embodiment, the first and third waveform signals are transmitted to the subject's nervous system. In another embodiment, the first waveform signal is transmitted proximate to a target zone on the neck, head, or spinal region.

According to the disclosed embodiments, the step of transmitting the waveform signals to a subject can be accomplished by direct conduction via attachment of an electrode to the receiving nerve or nerve plexus. As discussed, this requires a surgical intervention to physically attach the electrode to the selected target nerve.

In alternative embodiments, the step of transmitting the waveform signals to a subject can be accomplished by transposing the waveform signal into a seismic form. The seismic signal is then sent into a region of the head, neck, or spinal region in a manner that allows the appropriate “nerve” to receive and obey the coded instructions of the seismic signal.

The disclosed embodiments thus provide methods and apparatus to effectively control skeletal muscles. The methods and apparatus can thus be employed to restore some or most appendage (i.e., arm, hand and leg) movement in paralyzed subjects. The disclosed embodiments can also be employed in the treatment of various skeletal muscle impairments or disorders such as involuntary muscle contractions resulting from hypertonia and spasticity.

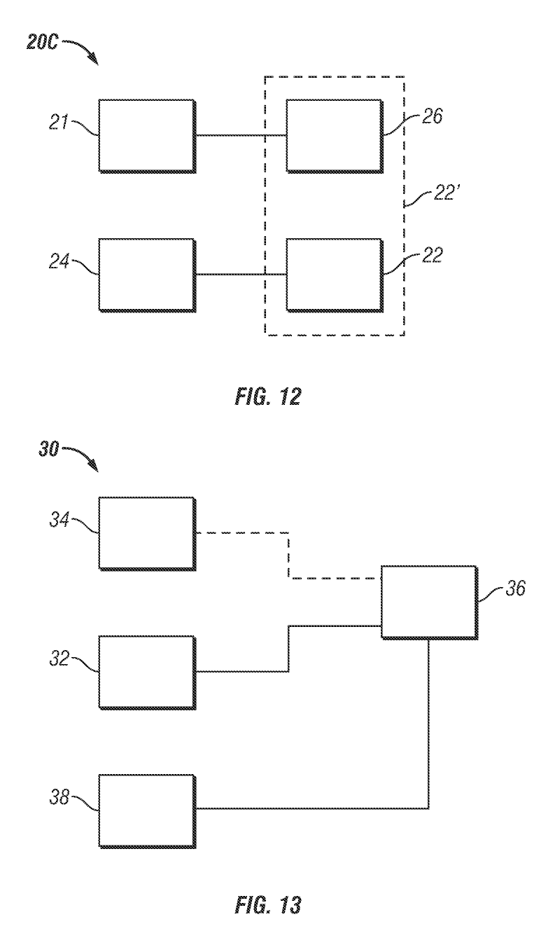

Referring now to FIG. 13, there is shown an embodiment of a skeletal muscle control system 30 that can be employed in the treatment of various skeletal muscle impairments and disorders. As illustrated in FIG. 13, the system 30 includes at least one skeletal muscle sensor 32 that is adapted to monitor the skeletal muscle function or status of at least a first skeletal muscle of a subject and transmit at least one signal indicative of the first skeletal muscle status.

According to the disclosed embodiments, the first skeletal muscle status can be determined by a multitude of factors, including skeletal muscle movement or lack thereof, muscle tension, etc. Various sensors can thus be employed within the scope of the embodiments to detect the noted factors and, hence, a skeletal muscle impairment or disorder.

The system 30 further includes a processor 36, which is adapted to receive the skeletal muscle status signal(s) from the skeletal muscle sensor 32. The processor 36 is further adapted to receive skeletal muscle waveform signals recorded by a skeletal muscle signal probe (shown in phantom and designated 34).

In a preferred embodiment, the processor 36 includes a storage means for storing the captured, waveform signals and skeletal muscle status signals. The processor 36 is further adapted to extract the components of the waveform signals and store the signal components in the storage means.

In a preferred embodiment, the processor 36 can be programmed to detect skeletal muscle status signals indicative of skeletal muscle impairments and/or disorders and/or waveform signals and components thereof indicative of skeletal muscle disorders and generate at least one waveform signal that is operative in the control of at least one skeletal muscle. Thus, in a preferred aspect of the noted embodiment, the processor 36 can be programmed to detect a skeletal muscle status signal indicative of a first skeletal muscle disorder and generate at least a first waveform signal that is operative in the control of the first skeletal muscle, which, when transmitted to the subject (as discussed below), mitigates the first skeletal muscle disorder.

Referring to FIG. 13, the generated waveform signal can be routed to a transmitter 38 that is adapted to be in communication with the subject's body. The transmitter 38 is further adapted to transmit the waveform signal into the subject's body (in a similar manner as described above) to control the affected skeletal muscle and, preferably, mitigate the detected skeletal muscle disorder.

According to the disclosed embodiments, the waveform signal is preferably transmitted to one or more nerves that are in communication with the affected skeletal muscle. A single waveform signal or a plurality of signals can be transmitted in conjunction with one another.

Thus, in accordance with a further embodiment, the method for controlling skeletal muscles generally comprises (i) monitoring the status of at least a first skeletal muscle of a subject, (ii) providing at least one skeletal muscle status signal in response to a skeletal muscle disorder of the first skeletal muscle, (iii) generating at least a first waveform signal that is operative in the control of the first skeletal muscle in response to the skeletal muscle status signal, and (iv) transmitting the first waveform signal to the subject to mitigate the skeletal muscle disorder.

In accordance with yet another embodiment, the method for controlling skeletal muscles generally comprises (i) capturing waveform signals that are generated in the body and are operative in control of skeletal muscles, the waveform signals including at least a first waveform signal that is operative in the control of a first skeletal muscle, (ii) monitoring the skeletal muscle status of the first skeletal muscle of a subject and providing at least one skeletal muscle status signal indicative of the status of the first skeletal muscle, (iii) storing the captured waveform signals and skeletal muscle status signal in a storage medium, (iv) generating at least a first waveform that is operative in the control of the first skeletal muscle in response to a skeletal muscle status signal or component of a captured waveform signal that is indicative of a skeletal muscle disorder, and (v) transmitting the first waveform signal to the subject to mitigate the skeletal muscle disorder.

In yet another embodiment, the method to control skeletal muscles generally comprises (i) capturing a first plurality of waveform signals generated in the body that are operative in the control of skeletal muscles, (ii) capturing at least a first waveform signal from a subject's body that is indicative of a skeletal muscle disorder, (iii) generating a confounding signal that is operative to mitigate the skeletal muscle disorder, and (iv) transmitting the confounding waveform signal to the subject to mitigate the skeletal muscle disorder.

As will be appreciated by one having skill in the art, the disclosed embodiments provide numerous advantages. Among the advantages are the provision of a system, apparatus, and method to control skeletal muscles that can be readily and effectively employed in the treatment of various skeletal muscle impairments and disorders, including involuntary muscle movement (e.g., spasms and muscle contractions) and partial or full loss of muscle movement or control resulting from spinal injuries, multiple sclerosis, cerebral palsy, radiation-induced nerve damage, stroke induced neuron damage, military warfare injuries resulting from bullets or explosions, etc.

Note that reproductions of the actual natural nerve codes that operate and modulate or regulate the movement of many muscles of the thoracic limb and other components are discussed herein. Eight signals that operate along with specific nerves to accomplish specific movements are presented herein. Each of the signals recorded from the various nerves possesses a use related to greater or lesser movements of the target muscles or muscle train.

In general, neuro-electrical signals can be sent from the brain to specific muscles via the nerve traveling to the muscles involved. The “brain command” is the initiator to operating the muscles. These natural signals vary by the type of neurons participating in the signal. There are probably some 300 types of neurons. A neuron is a cell with the capacity to generate a repeatable electrical signal that can be referred to as an “action potential” or “spark”. The neuron can be thought of as a rechargeable battery with the capacity to repeatedly generate an electrical action potential. The instantaneous recharge of any neuron occurs by means of a nutrient exchange from the blood stream and the extra cellular fluid, to prepare the neuron for firing another signal. Each type of neuron has the ability to fire a specific signal unlike that of any other kind of neuron.

A series of different neurons constitute a method to produce an electrical neuro-coded signal that carries instructions to a muscle to contract in a specific manner. Signal components may vary regarding the frequency and amplitude of the nerve signals to permit a given muscle to perform its particular duty. Each nerve includes a preferred combination of neuron types that make up its signal. The signals can be thought of as a combination of musical instruments with each different nerve playing a unique tune (code). Although using an inappropriate code on a nerve may initiate some particular action, the resulting action will not be as smooth or accurate as an action resulting from the code that belongs to a specific nerve, or it may not function at all.

The usual animal equivalent of a shoulder arm, forearm, hand, and fingers is the thoracic limb with a paw or long finger-like toes with claws rather than manipulative fingers as found in humans and monkeys. The un-diseased and uninjured animal uses the thoracic limb as a leg for movement from place to place plus uses it for handling of food, digging, and fighting. It should be noted that pets and zoo animal can suffer the same kind of injuries that humans suffer resulting in the loss or impairment of a limb. Thus, animals may also benefit from the disclosed embodiments, which can be utilized to operate specific muscles of the limb and paw.

Motor movements are impaired by disease or injury of muscles or by crushed or severed nerves leading to such muscles. Specific injuries to the brain, spinal cord or skeleton as well as lack of oxygen and other nutrients because of cardiovascular failure or damage to the respiratory tract can also lead to loss of proper limb control.

The ability to cause muscle movement(s) in humans or animals who have spinal or other injuries would be a welcome tool for the clinical physician or veterinarian. Nerve signals in human or animals could be controlled by the patient or a care-giver to allow graduated movement via muscular control with actual natural coded signals that replace those that are no longer available to operate the affected muscles.

Regarding muscles of the arm, hand, and fingers controlled by neuro-electrical signals, the neuro-coded signals are sent by the brain into the muscles via the radial, median, and ulnar nerves along with other selected nerves. The list of muscles provided herein which are operated by these nerves is not meant to be complete or all encompassing, but to provide a picture of the arena in which the disclosed embodiments, including alternative embodiments, operate. The location of these muscles can be ascertained and visualized in a standard or specialized medical anatomy atlas and/or a medical encyclopedia.

Muscles of the shoulder and the arm to the elbow include, for example, levator scapulae; pectoralae; deltoids; subcapularis; infra and supraspinatae; rhomboids and trapezius; coracobrachialis; brachialis; biceps and triceps brachii; and latisimus dorsi. Muscles of the hand, wrist, and forearm include, for example, finger digitorus extensor; supinator; finger and wrist flexors; pronator quadratus; pronator teres and biceps brachii; brachio radials; triceps brachii extensor indicis; extensor and flexor digitorum; flexor carpi ulnaris; abductor and flexor pollicis longus; thenar muscles; hypothenar muscles, and the central muscles of the hand.

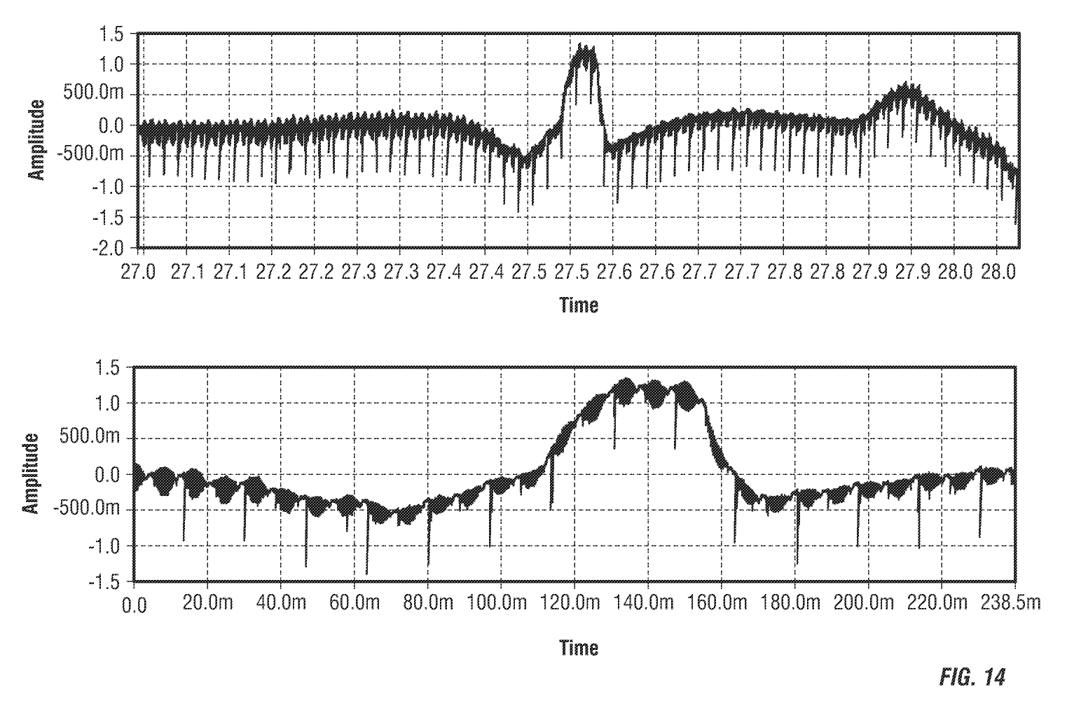

Embodiments include the actual natural neuro-coded signals that have been recorded from an animal or a human. It appears that a given code for a specific nerve should operate muscle across mammalian species. In other words, an animal code should also work in humans regarding any specific nerve/muscle combination to make more-or-less normal muscle function. An embodiment can be configured by previously recording the actual signals in the nerves leading to each of the skeletal muscles associated with the arm, forearm wrist, hand, and fingers in humans or animals and then rebroadcasting the signal to the preferred nerve and muscle to cause expected movements. FIGS. 14-22 herein illustrate a clear picture of the appearance of the signals. Note that the signals can be re-programmed to alter movement, intensity, and speed of movements.

The recording of the neuron generated waveform codes that operate the muscles may be accomplished utilizing means covered in, for example, U.S. Pat. No. 7,308,302, entitled “Device and method to record, store and broadcast specific brain waveforms to modulate body organ functioning,” which issued to Eleanor Schuler, et al on Dec. 11, 2007. U.S. Pat. No. 7,308,302 is incorporated herein by reference in its entirety.

Patients who suffer from paraplegia are largely victims of spinal injuries. Such patients can benefit from utilizing stored signals which can then be transmitted directly into the specific nerve(s) that move the afflicted muscle(s) and would not require transmission of the signal via the spinal cord. Signals can be coordinated via a small computer to operate one or multiple muscles to accomplish movement and to complete tasks. The neuro-coded signals operate at electrical energy levels at or near the energy levels of natural nerve signals to accomplish muscle movement in a controlled way. The operating voltage levels can be seen in FIGS. 14-22 herein.

Signals acquired from un-injured humans or animals are useful for medical treatment in those who are injured regarding nerve control of their upper limb muscles. Injuries that prevent transmission of neuron originated signals from the brain to a muscle have little treatments available to them from present day medicine. This applies to groups of individual muscles which need to be operated to accomplish an everyday task. Neuro-coded signals similar to those that previously operated the muscle can now be broadcasted into the appropriate nerves.

The electrical treatment output which carries the neuro-coded signals from the neuro-electrical code computer system has to be routed through output probes or devices. To send the codes into the nerve to operate a muscle requires one of two main engineering methods to transmit the signal in a manner that is acceptable by the nerve. The two methods are presented as “A” and “B”.

Method “A” involves directing output of the nerve electrical signal as the constant current method. This method requires that the current supplied by the neuro-electric computer system is consistent and repeatable throughout the delivery of the neuro-coded electrical signal. Using this method allows for the voltage level to fluctuate as the resistance changes. This method requires that a positive and negative probe (the negative probe located cranial to the positive probe) is attached to the nerve to be stimulated. The distance between the probes is 2 centimeters. A ground connection is made between the interior muscles and an earth ground.

Method “B” involves directing output of the neuro-electric coded signal as the constant voltage method. This method requires that the voltage supplied by the neuro-electric computer system is consistent and repeatable throughout the delivery of the neuro-coded electrical signal. This method requires that a signal probe is attached to the nerve to be stimulated. While the signal probe is capable of providing both the positive and negative portions of the neuro-code, only the positive portion of the neuro-code is used to stimulate. The signal ground probe is not required. A ground connection is made between the interior muscles and an earth ground. This method takes advantage of the magnetic/antenna properties of the probe and of the nerve.

The treatment coded signals have been reprogrammed from natural signals and therefore, may be applied via direct electrical connection by means of surgical attachment or they can be broadcast through the skin using focus magnetic devices capable of sending accurate signals to the appropriate nerves leading to the target muscles in a symphonic manner so that useful and appropriate movement and strength are accomplished.

Voltage changes for actual or reprogrammed nerve neuro-coded signals cause movement changes proportional to higher or lower voltage. For example, a neuro-electrical signal delivered at a slightly higher voltage will cause a stronger and larger muscle movement(s). Likewise, the same neuro-electrical coded signal delivered at a slightly lower voltage will cause a lesser and smaller movement of the target muscle(s). A given neuro-electrical signal for either a major or minor nerve with only voltage changes can be expected to cause different muscle movement activity which is repeatable using the same probe connection points and the exact voltage level.

Each nerve has a preferred neuro-electrical code to accomplish smooth naturally expected muscle movements. Voltages are generally less than 3 Volts. Much higher voltage levels with aberrant neuro-codes (those not preferred by the nerve) may make some muscle movement, but are not nearly as exacting and smooth as the more natural exact neuro-coded signals. An improper code tends to cause harsh unnatural muscle movements. It is expected that most mammals share specific codes for specific nerves.

During the process of recording or conducting neuro-electrical signals for the shoulder, arm, forearm, hand, and/or fingers the use of electrical grounding mats, grounding straps, and the ground connection with the interior muscles are required to minimize electrical noise and make handing of the nerve signals more exact. The grounding straps, mats, and any grounding connections regarding the neuro-electric code computer system are to be electrically connected to the same earth ground for proper results. Other grounding strategies may be utilized with actual patients who are able to move around using portable signal sources.

The disclosed embodiments thus include the discovery of the actual neuro-coded signals for causing functionality to the muscles of the shoulder, arm, forearm, hands, and fingers. Embodiments include the causing of movement in all of the joints concerned with the above anatomical structures. Shoulder, elbow, wrist, and finger digits movement are also included in the claimed movements. Altering the voltage up or down modulates or regulates the movement power, but does not alter the task function. Altering the voltage along with the time, forms a somewhat different signal as regards to a particular nerve.

FIG. 14 illustrates the neuro-electrical coded signal that operates the radial nerve in the arm, forearm, hands, and fingers. The signal brings the arm upward and the hands pull back with the fingers spread wide. This signal produces a more muscular and stronger performance as it makes a full movement.

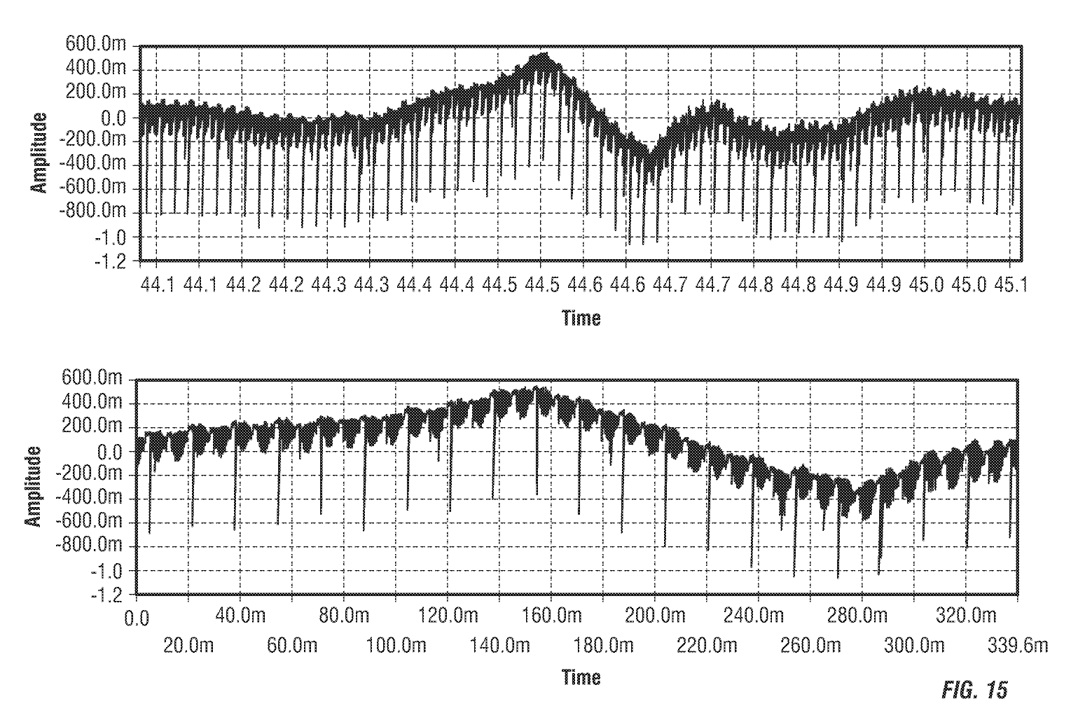

FIG. 15 illustrates the neuro-coded signal for raising the arm with the hand pulling backward and the fingers spread in a medium sort of movement. Note that the neuro-coded signal depicted in FIG. 14 offers a softer and somewhat lesser intensity of movement and renders somewhat of a medium movement compared to those described in FIG. 15.

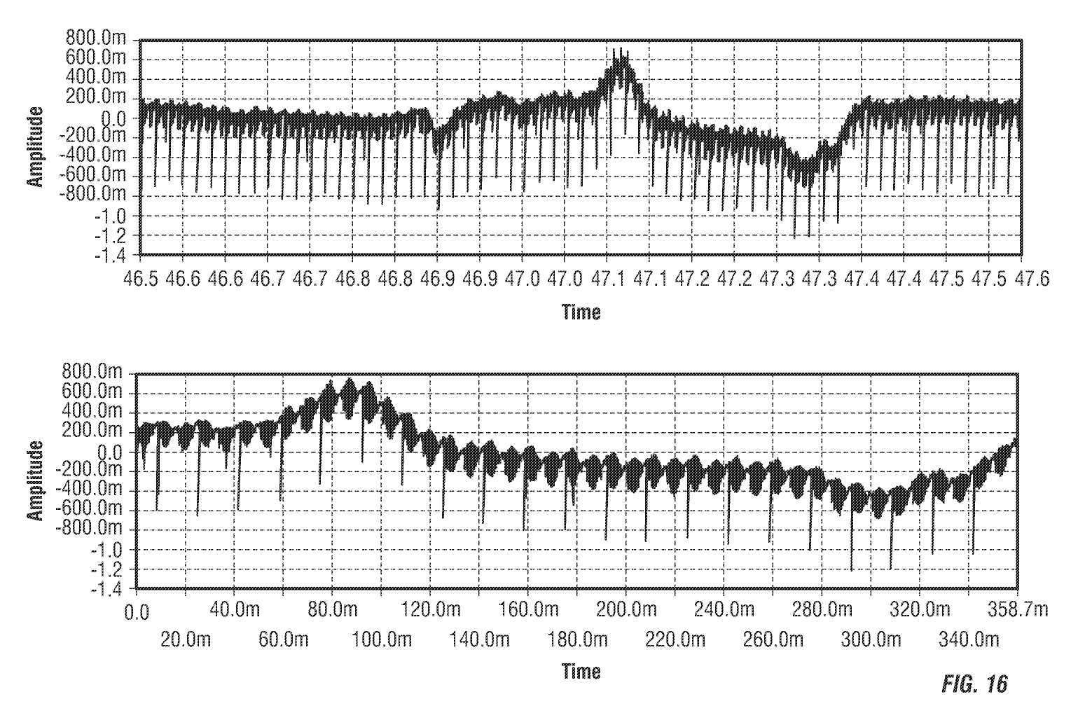

FIG. 16 illustrates the signal for the radial nerve that produces smaller and finer forearm movement consisting of lifting the arm up and twitching the hand backwards and to provide minor finger spreading. The signal depicted in FIG. 16 produces a more refined delicate movement of the muscles operated by the radial nerve.

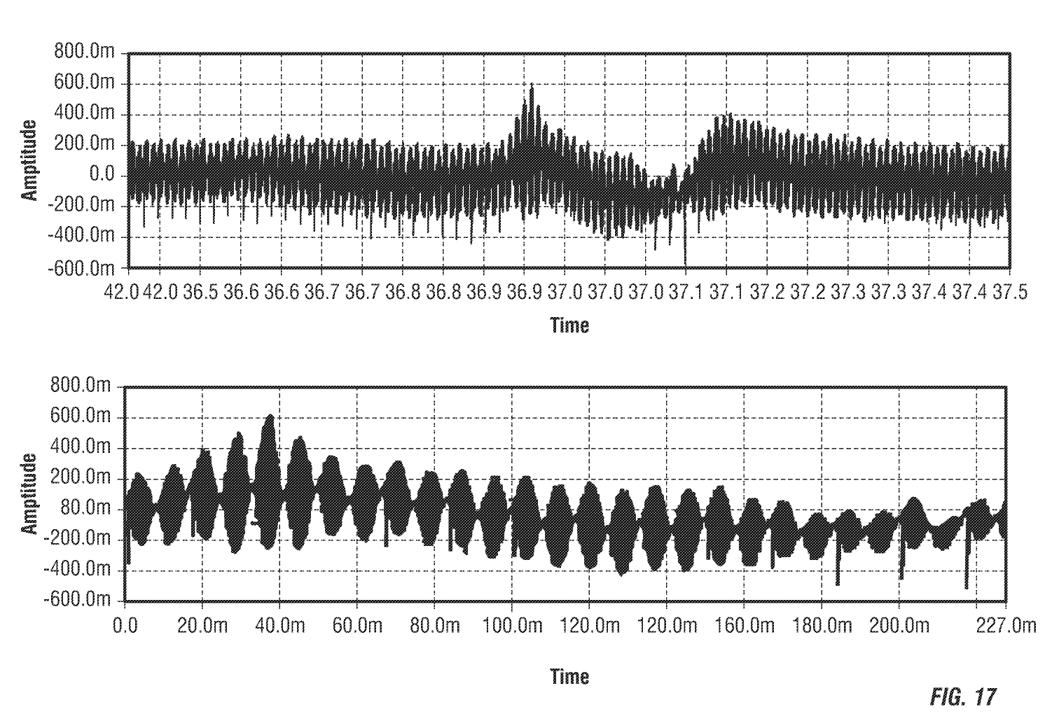

FIG. 17 illustrates the neuro-coded signal that provides large powerful movements in muscles served by the ulnar nerve. This signal moves the arm and forearm downward, pulls the fingers together, and makes a fist.

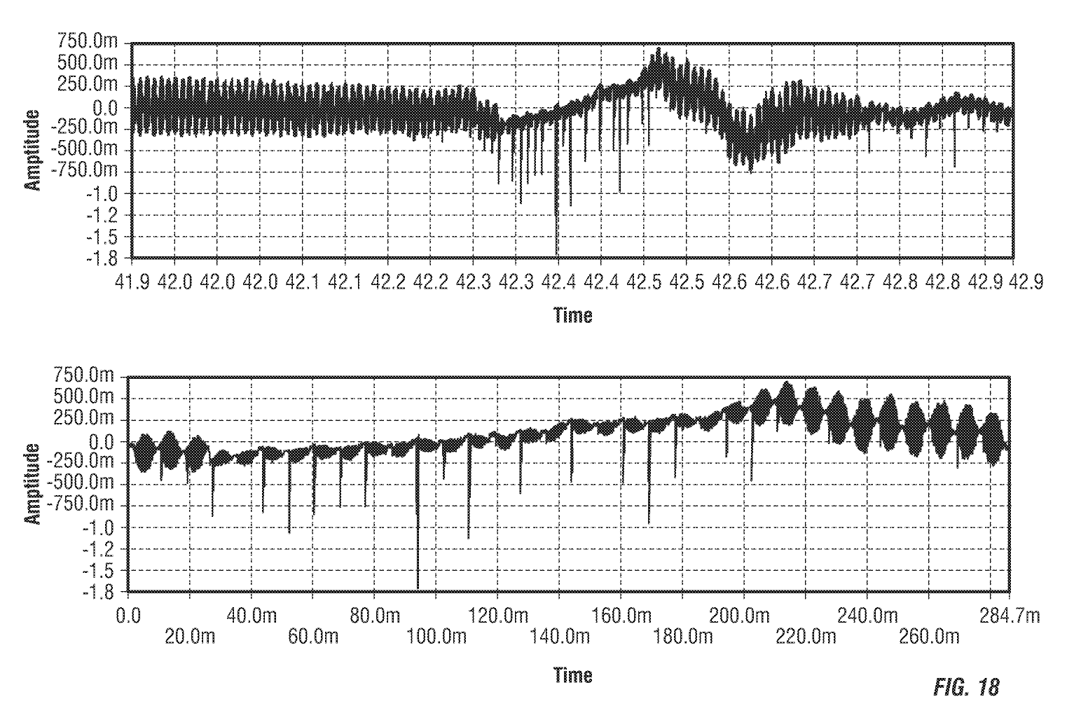

FIG. 18 illustrates a neuro-coded signal, which demonstrates and causes the movement via the ulnar nerve of the forearm movement downward with wrist flicks downward and minor finger movements.

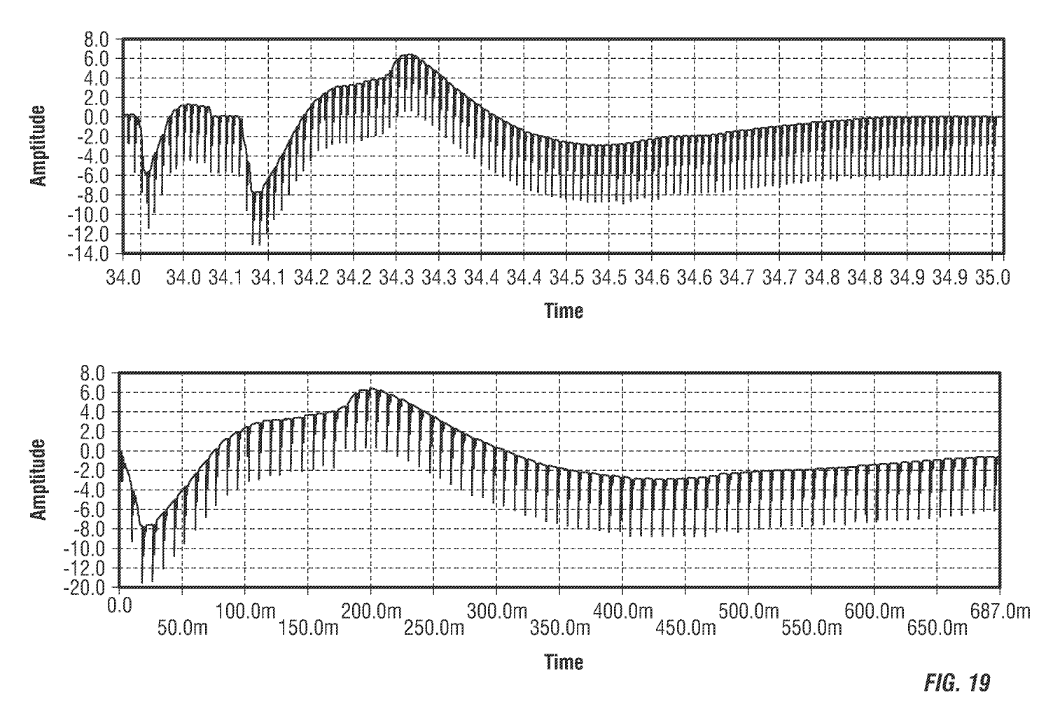

FIG. 19 illustrates the signal charged with making smaller movements via the ulnar nerve to appropriate muscles of the forearm downward with smaller wrist twitches also in a downward direction.

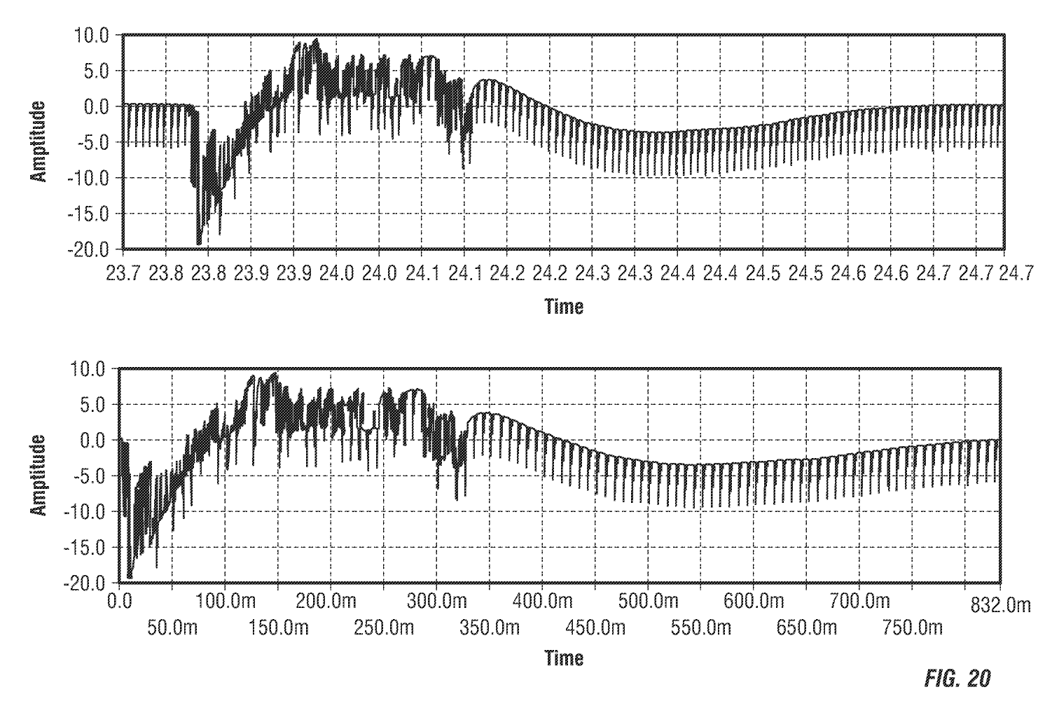

FIG. 20 illustrates the signal of the supra scapular nerve providing major movement of the arm by pulling upward and somewhat outward.

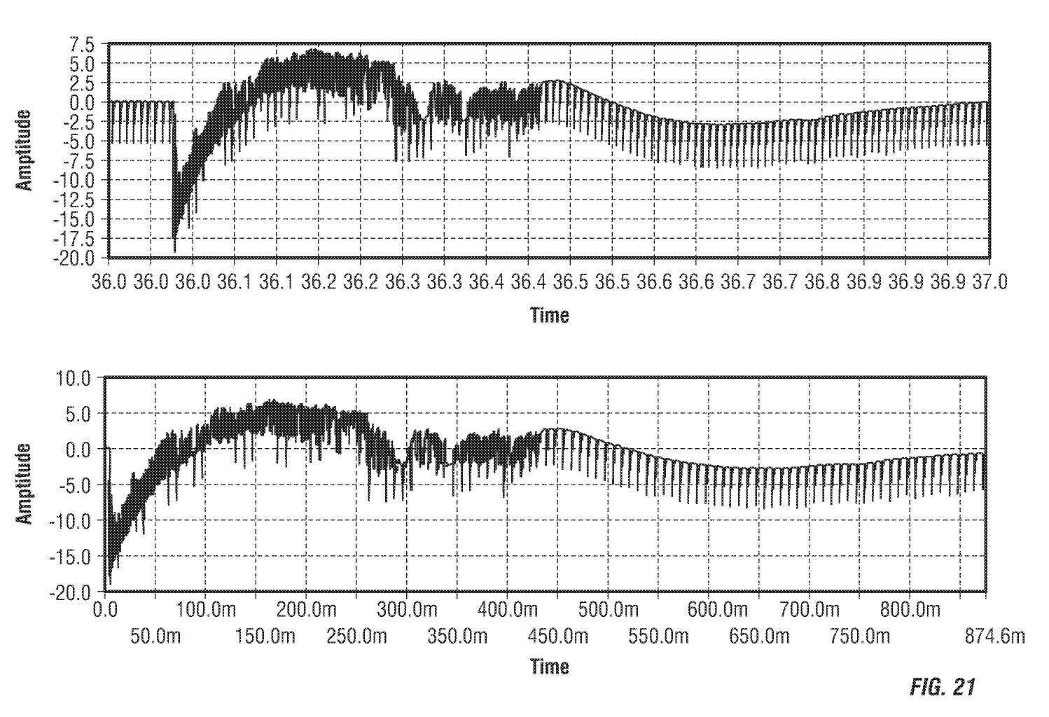

FIG. 21 illustrates the signal of the supra scapular nerve to activate moderate movement of the arm by pulling it in an upward and outward direction.

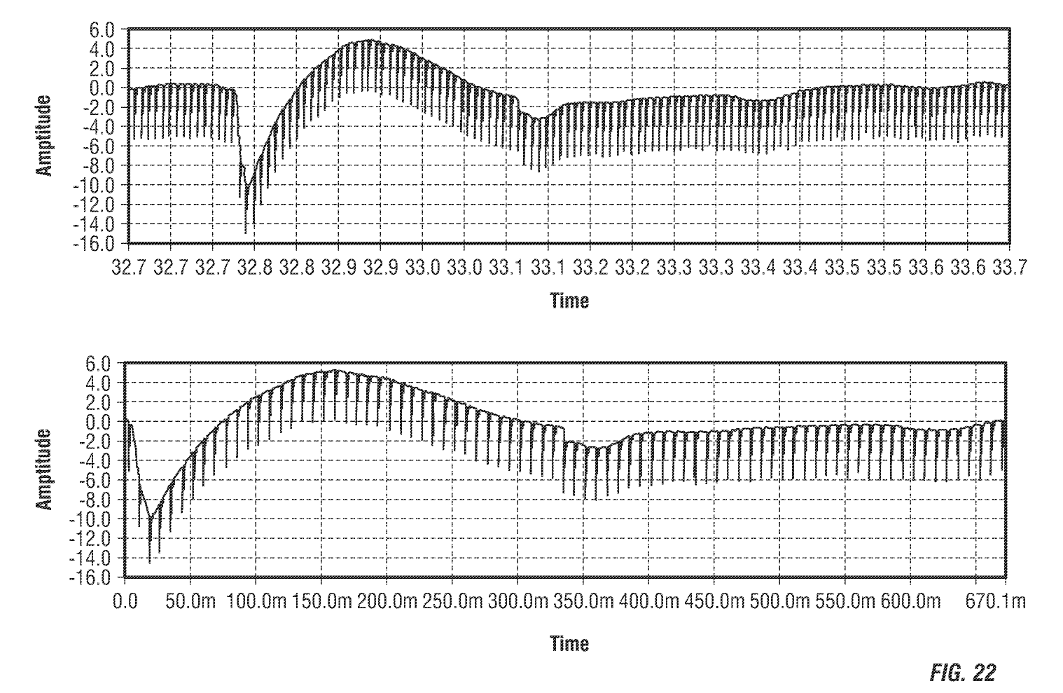

FIG. 22 illustrates the signal carried by the scapular nerve to move the arm and shoulder to work powerfully in a downward digging or pawing the ground motion.

The signals represented by FIGS. 14-22 are useful, with variable voltage, toward reproducing the expected natural movements of the shoulder, arm, forearm, hand, and fingers. Once the signals are recorded from a normal human or animal, they can be applied to the nerves of subjects who cannot transport the signals from the brain to the nerves that operate, as appropriate, the muscles of the shoulder, arm, forearm, wrist, hand, and fingers, including the thumb. Different muscle can be operated via the electrical neuro-coded signals to steer the limb as necessary to complete a desired task. Variation of the voltage causes the gradations in the various muscles to accomplish the duties required by the electrical neuro-coded signals.

The disclosed embodiments include using actual neuro-electrical coded signals or waveforms for application to humans or animals to adjust or control skeletal muscle movements for the shoulder, arm, forearm, wrist, hand, and fingers to accomplish work and coordinated tasks. The actual neuron generated signals cause and refine normal muscle signals. Such actual muscle signals can be regenerated from an internal or external place or point on or in the human or animal body for medical treatment so as to cause normal and more-or-less usual movement given the circumstances of the patient or subject.

In accordance with the disclosed embodiments, the natural signals can be electrically varied as to frequency and amplitude so as to cause normal fast or slow muscle movement as well as deeper or lesser movements to meet the demand of the animal or human brain. Additionally, a menu of actual muscular neuron coded signals may be cataloged. The system shall be able to modulate system output so as to fine-tune muscle movements. Some signals may be “all-or-none” so to speak, in sending activation or deactivation responses and other signals and may merely tweak a fine motor action or aspect to refine muscle tone or action.

In some embodiments, the treatment output voltage and amperage may be very low, but a larger electrical span can be employed to allow for any electrical resistance encountered. The voltage levels for the neuro-coded signals or waveforms from the treatment shall not exceed 3 volts or 1 amp in its output electrical energy for each channel of signal. Up to 10 or more channels may be used simultaneously to exert medical treatment on muscular control to aid a patient in moving or performing muscular tasks suitable to his or her well-being as medical treatment.

The actual waveform or neuron generated signals may have the appearance as shown in previous patent applications by the same inventors as described herein. Such a signal can be enlarged or reduced electrically as to amplitude and repetitive frequency. Such signals preferably do not exceed 3 volts or 1 amp in electrical configuration.

In accordance with the disclosed embodiments, the muscle operating signal occurs naturally as burst signal(s) followed by a pause and then another burst of neuronal activity followed by a pause and so it is on and on throughout life. The signal amplitude or time of pause can be varied to accomplish the muscle deed required. Muscle activity requires variable repetitive neuro-coded signals as humans or animals move their shoulder, arm, forearm, wrist, hand, or fingers. Various muscles operate in a symphonic pattern being conducted by the brain to accomplish the mission assigned There is adequate, but variable space between the signals to allow synchronization of movement into smooth actions.

There may be electrical or mechanically induced noise in the signal used for treating muscle disorders. These signals are a close approximation or the exact natural neuro-coded signal, but the formation of the signal may have to be electrically filtered or cleaned up to be more exact in appearance just like the natural codes. But it does represent the actual signal collected from the muscle of a human or animal that is not impaired so as to make it available to a patient who is impaired as to that same signal. It is claimed that such neuro-electric coded signals would be the same or closely similar in all mammals or humans.

In accordance with the disclosed embodiments, the gradation of muscle movement may be accomplished by slight or great changes in applied neuro-electrical coded voltage. Voltage changes, either higher or lower, make a variety of predictable graduated muscle movements possible, provided the correct and actual neuro-electric code is applied to the nerve(s) in order to control and operate the targeted muscle(s) so as to bring useful movement to the people or animals that suffer paralyzed shoulder, arm, forearm, hand, and/or fingers.

Note that a breakdown of the radial, ulnar, suprascapular, and scapular movements can be presented as follows:

### Radial

1-major forearm movement bringing arm up, hand pulls back, and fingers spread
2-forearm movement bringing arm up, hand pulls back, and fingers spread
3-smaller forearm movement bringing arm up, hand twitches back, and minor finger spread

### Ulnar

1-major forearm movement downward, wrist pulls down, and fingers come together into a fist
2-forearm movement downward, wrist flicks downward, and minor finger movement
   3-smaller forearm movement, wrist twitches downward

### C6 Suprascapular

1-2 medium to major movement of the arm, pulling upward, and slightly outward

### Scapular

1-3 medium to major movement of the arm in the motion of running or digging

The following data is also presented:
Increasing Voltage makes the movement full
Decreasing Voltage makes lesser (sp) movements
Lesser Seconds=greater movement
Longer Seconds=lesser movement

Note that the disclosed embodiments also include the disclosure of a method and system for controlling the movement and locomotion of lower limbs by means of neuro-coded electrical signals. Such a method and system includes utilizing ultra-low voltage neuro-coded electric signals to reactivate selected muscles in the region of the thigh and lower to accomplish useful gradated activation of all the lower limb muscles and to orchestrate muscle movement to accomplish activities such as walking, running, standing, bending, kneeling, twisting, or even standing on one's toes. The muscle activating signals that are conducted or broadcast into the muscles are ultra low voltage neuro-electric coded signals that are similar to natural signals. These medical treatment signals are stored within and generated from small computerized electronic devices that can systematically activate muscle groups to accomplish useful movement and locomotion in humans or animals that are impaired by neurological disease or injury.

A variety of trauma injuries or diseases affecting the spinal cord of humans and animals results in malfunction, partial function, or no function of the limbs. The nature of the injuries to the nerve fibers of the spinal cord occur by severing or crushing the nerves to the point where the electrical signals from the brain are distorted or unable to communicate with the various muscle groups that operate the limbs. There are a significant number of diseases that can affect the function of the lower limbs. These diseases can range from strokes that affect the motor cortex of the brain to demyelination conditions that result in loss of the nerve insulation such as in MS. Diseases of the brain or of the spinal column as well as muscle disease or injury have as an outcome paralysis for the owner of the limb. Crutches, braces, wheel chairs, or beds become part of the daily life of those with lower limb paralysis or lesser impairment.

Since most of the injuries or diseases that befall the nerves of the brain, spine, and lower muscle groups result in a full or partial loss of electrical signaling and signal reception in important muscle groups required for movement and locomotion. There is a need to recapture the ability to operate the lower limbs by restoring the ability to send and receive the neuro-coded signals required for effective operation of the lower limbs.

Muscles of the hips, thighs, knees, legs, ankles, and toes are some of the operational components that make up the lower limbs. All of the useful movement in these structures happens because of coded neuro-electric signals traveling from the brain and down the spinal cord via the nerves to arrive at individual muscles to provide the instructions for useful movement.

The disclosed embodiments are generally concerned with controlling the muscles of the lower limb(s) that in every-day life are concerned with walking, running, standing, and balancing of the body. The lower body is the carriage that carries the upper body and brain in a direction that the mind has selected. There is no need to send the neuro-coded signals down the spinal cord. The signals can be applied directly to peripheral nerves that lead to activate any of the muscles of the lower limb to include the muscles involved in locomotion and movement of any and all parts of the lower limbs.

Natural locomotion of the muscles of the lower limbs are largely controlled by ultra-low voltage nerve signals that originate in the brain and travel along at an energy of one (1) to two (2) volts at millionths of an amp. Neuro electric coded signals cause gradated contraction of the large and small muscles of the lower limb region.

The Sciatic nerve is the principal nerve for operation of the leg in animals and humans. It branches off into other nerves, most notably the common peroneal segment and the tibial segment. The tibial nerve and the plantar nerve play a major role in control of the foot, including the great toe which is so important in maintaining balance. There is also the obturator nerve that regulates abduction and rotation of the thigh. The femoral nerve contributes to the flexing and inward rotation of the lower leg as well as extension of the knee joint region. The femoral nerve plays a major control role for the quadriceps. There are cutaneous nerves and afferent sensory branches that supply sensation of the skin and positional information to the brain. Afferent sensory branches are found with efferent major and minor nerves which supply action and contraction to all of the muscles concerned with contracting, flexing, rotating, bending, extension and to do this in harmony to cause smooth, powerful or delicate movements in all of the muscles concerned with the lower limbs.

The duties of the leg and foot also rely on the deep peroneal nerve, fibular nerve, superficial fibular nerve, and of course the tibial nerve. The term “leg” actually can be defined as the structure between the knee and the ankle. In practical use, however the leg seems to refer to the structures that emerge from the pelvis as the thigh, on to the knee, and the lower leg, but not to include the actual foot and toes. Note that the entire limb from the hip and thigh on to the knee area, leg, and on to the toes can be considered players in walking, standing, and balancing of the entire body.

The muscles of the thigh and hip are activated by a number of nerves, the sciatic being the most important with many conductive fibers that activate many aspects required to operate the limb. Nerves split off, branch, and merge into other nerves to accomplish locomotion. Different names have been applied to various aspects of the network of nerves that activate the various muscles of the entire limb.

Locomotion of the leg is possible via neuro-coded signals emanating from the brain which are carried by the peripheral nerves of the sciatic and its branches. Without nervous control it is impossible to control and operate the leg so as to walk in all mammals. The sciatic nerve has branches identified as peroneal segment of sciatic nerve, the common peroneal nerve, the tibial segment of the sciatic nerve, and the tibial nerve. These nerves are neuro-coded operated by sending and receiving neuro-coded signals from the brain and brainstem to the various segment of the sciatic nerve. The sciatic nerve emerges from L4, L5, S1, S2, and S3 of the spine.

The nerves of upper lower limb (thigh) emerge from L1, L2, L3, and L4 of the spine and contain two nerve plexuses which are jointly referred to as the lumbo-sacral plexus. The two principal nerve branches traveling into the leg are the femoral and obturator. The sacral plexus enervates the buttocks and continues downward as the sciatic nerve. The inferior gluteal nerve is involved in the control of the gluteus maximus, the most coarsely fibered and heaviest muscle in the body, and forms the great bulk of the buttock. It extends and rotates the hip joint which involves running, standing or climbing stairs. The gluteus maximus is used in standing up from a sitting position.

Large bones support the body, provide muscle and ligament attachment points, and allow the bipedal locomotion (walking and running gait) familiar in humans. These major long bones are the femur, tibia, and fibula. The numerous bones of the ankle and foot which are essential for all movement and balance activities are composed of the calcaneus, cuboid, metatarsal, phalanges, talus, cuneiform, and sesamoid and phalanges bones.

The leg is a made up of fibrous muscle which is activated by the neuro-coded signals emanating from the brain. For walking and movement of the leg, the muscles of the leg must be electrically activated by the various nerve branches comprising the sciatic nerve. This activation by nerve fibers containing both efferent nerve fibers that travel from the brain to cause the various muscle to contract and the afferent fibers that report to the brain on the status of muscle movements and positional status of the leg. By regulating the firing of contractile neurons attached to fibrous muscle, gradated movements can occur.

The neuro-coded signals concerning skeletal muscle movement and control originate in higher brain structures. The neuro codes emerge from the brain and travel to nerve fibers of the spinal column where they travel within the spinal column to emerge from the lumbar and sacral areas through pelvic greater sciatic foramen (natural opening in bone) and then into the thigh and leg sciatic enervation.

The sciatic nerve branches and splays into the various muscles of the leg. The muscles of movement and locomotion are fully enervated so as to be able to activate the large area of contractile fibrous muscle. A neuro code burst, traveling down the sciatic and related nerves, spreads throughout the leg muscles to activate movement. The arrival of the neuro-code activation causes contraction of the muscles in a controlled way to accomplish movement as required by the owner.

When the neurons stop their neuro-code electrical bursting, the leg movement stops and the leg relaxes. Thusly, leg movement and muscle rest is accomplished as a direct influence of neuro-coded signals traveling on the sciatic nerve. Continuous locomotion is accomplished in the human and animal body by continuous bursts of neuro-coded signals. The signals are similar in nature for small or large muscle activity. The changes in gradated leg movement performance can be accomplished by variations of approximately 20% up to 70% in voltage amplitude of the signals. Frequency of a given movement is accomplished by the time spacing of the neuro coded signal bursts.

The actual sciatic nerve signals have been recorded, stored, and rebroadcasted onto the sciatic nerve to accomplish leg movement. The neuro-coded signals can operate at a range of voltage from with millivolts of power (thousandths of a volt) up to about 2 volts.

The disclosed embodiments also provide a method and system for activating (e.g. turning on) the neuro-electrical signals that cause controlled major and minor movements in the muscles of the lower limbs. Such embodiments may be employed for medical treatment use in individual animals or humans who have lost the use of a muscle or group of muscles, or who have suffered a spinal injury or disease that prevents them from locomoting.

Neuro electric coded signals are captured from a plurality of mammals for all of the mammalian muscles from the waist downward that are involved in standing, leaning, walking, running, or balancing in the up right or other positions. The signals can be used as medical treatment in a plurality of humans or animals that require activation of locomotion and balancing muscles to improve their life and health.

The captured signals can be stored in a memory of a data-processing apparatus and then processed and adjusted by the data-processing apparatus for application to each muscle that is impaired. The orchestrating data-processing apparatus (e.g., computer) can be utilized to organize the individual codes so as to perform the locomotion or standing or balancing movement. Individual electrodes or transmitting devices are hooked or brought into close proximity to each muscle that is to be activated.

As a practical matter, it can be expected that additional voltage up to, for example, 3 volts may be required to allow signal transport through any natural nerve insulation or resistance at the connection before it reaches the neurons that originate and relay the signal for actual leg muscle movement and to include walking, running, kicking, and for lifting heavy objects.

In accordance with another embodiment, a neurological signal scientific computer system may be employed to collect and store the actual coded signals produced by mammalian brains and brainstem. Such stored signals can then be broadcast onto or into the sciatic nerve and its branches to activate and regulate the muscle of the leg. The amount of movement or locomotion and the frequency of movement are controllable with variations in the neuro-coded signals.

An embodiment may also provide a system and method for broadcasting the leg motion neuron-codes previously collected from a mammal's respiratory tract. Such neuro-codes are the neuron generated codes that travel from the brain and brainstem onto and within the nerves of the leg. Neuro-coded signals coming from the brain and brainstem are the effector signals (efferent) and the signals coming from the sciatic nerve travel to the brain are the afferent or sensory nerves which report on the status of leg muscle movement. The activation and operation of the leg function fully depends on the signals traveling on and within the sciatic and associated nerves.

The neuro-coded treatment signals, which are similar to the natural signals, can be used to activate the muscles of the leg via the sciatic and other nerves concerned with the lower limbs of humans or animals. The neuro-electrical activation sequencing of the muscles are controlled by miniaturized computerized devices that carry the neuro codes and have output electrodes for attachment to the muscles. The neuro-signal voltage can be adjusted by the user or can run a computer program for various functions on the order of millivolts up or down to regulate larger or smaller leg muscle movements to accomplish running, walking, or lifting of heavy objects and any other required or desired movements of the muscles of the lower limb. Note that in some embodiments, the neuro coded signals, which are similar to natural signals, may be applied to the peripheral nerves that control the muscles of the lower limb.

Note that the disclosed embodiments can include aspects as discussed in the above referenced patent applications related to the ability of the neuro-code computer to record, store, and conduct signals into the selected nerves. Such U.S. patent applications incorporated by reference herein as indicated above, describe background information related to recording, storing, adjusting, and broadcasting the nerve signals to the appropriate peripheral nerves so as to accomplish the neuro-coded signal movement of the thigh, leg, ankle, and foot, among other muscles.

The treatment orchestrating computer and muscle connection apply neuro-electric codes that have been recorded from a plurality of animals or humans into the computer or other data-processing apparatus, wherein the signals are sorted and adjusted for use as medical treatment. The selected codes can then be utilized to provide medical treatment in a plurality of mammalian patients.

The orchestrating computer can be utilized to adjust the neuro-electric coded signals so as to accomplish the locomotion or balancing required by the human patient, who may perform the adjustments or they may have been pre-programmed by the physician. Animals may require veterinarian programmed computers to attain the desired movement.

One potential embodiment includes a method and system for generating and controlling neuro-coded signals to be used to transmit, conduct, or broadcast such signals onto peripheral nerves that operate the muscles of the lower limb. Such an embodiment may be utilized for the medical treatment of mammals. Another embodiment includes a method and system for generating coded signals from a computerized battery operated device that can conduct or broadcast such signals into the muscles of the lower limb to cause and initiate movement and locomotion. Such an embodiment may be employed for use with fully or partially paralyzed humans or animals. An additional embodiment includes a method and system for utilizing certain neuro coded signals that can operate the sciatic and other nerves to activate any of the muscles of the lower limbs to cause graduated movement of any selected muscle so as to assist in operating such lower limb. These signals have the capacity to make small or larger muscle movements with slight variation of signal voltage. Voltage range spans millivolts up to 5 volts to accomplish movement of any of the muscles involved in movement of the muscles of the lower limb.

Note additionally that the neuro coded electrical signal, which has a voltage in the millivolt range can be adjusted as to amplitude up or down, to accomplish changes in the degree of leg muscle movement. In no case does the voltage exceed 3 volts to perform useful work with the leg muscles. Frequency of leg movement can be adjusted via the scientific neuron code computer system to call for signals that provide the selected ultra low voltage neuro-coded signal for lower limb near natural motion and movements.

The treatment signals can be acquired and recorded from a plurality of animals or humans and stored in a scientific computer or other data-processing apparatus. Once stored, the signals can be adjusted and programmed for the desired treatment. Treatment can be applied to a plurality of humans and animals to correct or assist locomotion and balancing by activating, in a programmed manner the orchestration of signal application. Computer technology can be applied to the orchestration of signal application.

Based on the foregoing, it can be appreciated that a method and system are disclosed for regulating and controlling the movement of skeletal muscles that operate the upper and lower limbs of humans and animals by means of neuron encoded signals of nerves. In general, such a method and system can include collecting and storing analog nerve neuro-signals of both injured and normal skeletal muscles from any part of the body of a human or animal subject with a system consisting of a neuro-code processing scientific computer system, known as a Neuriac®; collecting and recording such neuro-signal(s) from a nerve leading to a selected skeletal muscle by means of a metal electrode attached to an analog to digital converter which leads to a digital processing unit within a Neuriac® (such signal(s) can be stored and then subjected to reprogramming if required into a treatment waveform composed of groupings of neuron signals); selecting at least a first skeletal muscle signal from a library of stored skeletal muscle waveforms within the Neuriac® or other neuro-signal(s) scientific computer system that can record, store, reprogram, and transmit treatment signal(s); and transmitting or conducting the waveform to one or more nerves that operates or regulates at least one skeletal muscle. The subject may then adjust the waveform signals to cause the muscle to perform as intended.

The approach described herein can provide for the regulated movement of skeletal muscles in a smooth and natural manner for body parts such as, for example, hands, wrist and fingers, upper arm, lower arm, shoulder, upper leg and lower leg including the ankle and toes, among many other skeletal muscle groups throughout the human and animal body. In some instances, a waveform signal can be transmitted to the subject's nervous system to cause smooth operation of at least one skeletal muscle. In other embodiments, a plurality of neuro-signal waveforms, which are similar to waveforms generated in the body, can be simultaneously and/or synchronized during transmission to a plurality of muscles to operate a complex of muscles such as found in a hand.

Additionally, a series of reprogrammed neuro-signals can be loaded into a small portable programmable treatment device to be attached to a subject's body and connected to a selected skeletal muscle group. Said device can be turned on or operated by the subject to transmit a series of neuro-coded signals for operation of a body part. Such a body part may be signaled to close the fingers and position the thumb to pick up an object, for example.

In still other embodiments, a method and system can be implemented to ensure that a full limb system becomes operable by the subject to control a leg or arm that is partially inoperable due to a spinal cord injury. A portable programmable device can be provided wherein treatment signals are transferred from a Neuriac® system into the portable treatment device. Neuro-electric connections can be made throughout the limb so that all the desired movements are operable. The subject may turn-on and command the control aspects of the limb as is desired with a hand held or attached remote control device. In yet another embodiment, a method and system can be implemented, which includes a wheel-chair mounted keyboard controller that generates pre-programmed skeletal muscle signals to a plurality of pre-attached neuro-electrodes designed to operate all 4 limbs to the extent that the health of each limb allows.

It will be appreciated that variations of the above-disclosed and other features and functions, or alternatives thereof, may be desirably combined into many other different systems or applications. Also, that various presently unforeseen or unanticipated alternatives, modifications, variations or improvements therein may be subsequently made by those skilled in the art which are also intended to be encompassed by one or more of the disclosed embodiments. Of course, it can be appreciated that all neuro-coded signals, be they natural skeletal muscle encoded signals or reprogrammed treatment signals, are always analog. The programming of treatment signals, however, may be accomplished via a digital computer process or after which the signals are converted from digital to analog and prior to applying any treatment signal to at least one skeletal muscle to cause controlled movement(s).

What is claimed is:

1. A method for regulating and controlling the movement of skeletal muscles that operate the upper and lower limbs of humans and animals by means of neuron encoded signals of nerves, said method comprising:
collecting and storing, utilizing a data-processing apparatus, at least one nerve neuro-signal of both injured and normal skeletal muscles from any part of a body of a human or an animal subject, said at least one nerve neuro-signal initially in an analog format;
collecting and recording said at least one nerve neuro-signal from a nerve leading to a selected skeletal muscle via a metal electrode attached to an analog-to-digital converter that communicates with a digital processing unit associated with said data-processing apparatus that stores and re-programs, if required, said at least one nerve neuro-signal into at least one treatment waveform composed of groupings of neuron signals;
selecting at least a first skeletal muscle signal waveform from a library of stored skeletal muscle waveforms stored in a memory of said data-processing apparatus, wherein said data-processing apparatus is configured to record, store, reprogram, and transmit said at least one treatment waveform; and
transmitting or conducting said at least one treatment waveform from among a plurality of neuro-signal waveforms to at least one nerve that operates or regulates at least one skeletal muscle.

2. The method of claim 1 further comprising adjusting said at least one treatment waveform to cause said at least one skeletal muscle to perform as intended.

3. The method of claim 1 further comprising adjusting said waveform to provide for a regulated movement of said at least one skeletal muscle in a smooth and natural manner for at least one of the following of said body: hands, wrist and fingers, upper arm, lower arm, shoulder, upper leg and lower leg including the ankle and toes.

4. The method of claim 1 further comprising transmitting a first waveform signal to a nervous system of said body to cause a smooth operation of said at least one skeletal muscle.

5. The method of claim 1 further comprising:
providing said plurality of neuro-signal waveforms, wherein each waveform among said plurality of neuro-signal waveforms is similar to waveforms generated in said body; and
simultaneously transmitting each waveform among said plurality of neuro-signal waveforms as treatment waveforms to a plurality of muscles to operate a complex of muscles of said body.

6. The method of claim 1 further comprising:
providing a portable treatment device attached to said body and connected to a selected skeletal muscle group of said body;
loading a series of re-programmed neuro-signals into said portable treatment device; and
transmitting at least one re-programmed neuro-signal into said series of re-programmed neuro signals from said portable treatment device to said selected skeletal muscle group for operation of a body part associated with said selected skeletal muscle group.

7. The method of claim 1 further comprising:
transferring said at least one treatment waveform from data-processing apparatus to a portable programmable device;
providing neuro-electric connections from said portable programmable device to and throughout a limb of said body so that all desired movements of said limb are operable; and
permitting a user to command and control aspects of said limb utilizing a hand held control that communicates with said portable programmable device.

8. The method of claim 7 further comprising:
providing a wheel-chair mounted keyboard controller that transfers at least one pre-programmed skeletal muscle signal of said body to a plurality of pre-attached neuro-electrodes that operate all four limbs of said body to an extent that health of each limb among said four limbs allows.

9. The method of claim 1 wherein said data-processing apparatus comprises a neuro-code processing scientific computing system.

10. A system for regulating and controlling the movement of skeletal muscles that operate the upper and lower limbs of humans and animals by means of neuron encoded signals of nerves, said system comprising:
a data-processing apparatus for collecting and storing at least one nerve neuro-signal of both injured and normal skeletal muscles from any part of a body of a human or an animal subject, said at least one nerve neuro-signal initially in an analog format;
a metal electrode attached to an analog-to-digital converter that communicates with a data-processing unit of said data-processing apparatus, wherein said at least one nerve neuro-signal is collected and recorded from a nerve leading to a selected skeletal muscle via said metal electrode attached to said analog-to-digital converter that communicates with said digital processing unit associated with said data-processing apparatus, wherein said data-processing apparatus that stores and re-programs, if required, said at least one nerve neuro-signal into at least one treatment waveform composed of groupings of neuron signals;
a library of stored skeletal muscle waveforms, wherein at least a first skeletal muscle signal waveform is selected from said library of stored skeletal muscle waveforms stored in a memory of said data-processing apparatus, wherein said data-processing apparatus is configured to record, store, reprogram, and transmit said at least one treatment waveform; and
a transmitter for transmitting or conducting said at least one treatment waveform from among a plurality of neuro-signal waveforms to at least one nerve that operates or regulates at least one skeletal muscle.

11. The system of claim 10 wherein said at least one treatment waveform is adjustable to cause said at least one skeletal muscle to perform as intended.

12. The system of claim 10 wherein said waveform is adjustable to provide for a regulated movement of said at least one skeletal muscle in a smooth and natural manner for at least one of the following of said body: hands, wrist and fingers, upper arm, lower arm, shoulder, upper leg and lower leg including the ankle and toes.

13. The system of claim 10 wherein said transmitter further transmits a first waveform signal to a nervous system of said body to cause a smooth operation of said at least one skeletal muscle.

14. The system of claim 10 wherein:
each waveform among said plurality of neuro-signal waveforms is similar to waveforms generated in said body; and
each waveform among said plurality of neuro-signal waveforms is simultaneously transmitted as treatment waveforms to a plurality of muscles to operate a complex of muscles of said body.

15. The system of claim 10 further comprising:
a portable treatment device attached to said body and connected to a selected skeletal muscle group of said body, wherein a series of re-programmed neuro-signals is loaded into said portable treatment device; and
wherein said transmitter transmits at least one re-programmed neuro-signal into said series of re-programmed neuro signals from said portable treatment device to said selected skeletal muscle group for operation of a body part associated with said selected skeletal muscle group.

16. The system of claim 10 further comprising:
a portable programmable device, wherein said at least one treatment waveform is transferred from said data-processing apparatus to said portable programmable device;
wherein said neuro-electric connections are provided from said portable programmable device to and throughout a limb of said body so that all desired movements of said limb are operable; and
a hand held control that permits a user to command and control aspects of said limb utilizing said hand held control, wherein said hand held control communicates with said portable programmable device.

17. The system of claim 16 further comprising:
a wheel-chair mounted keyboard controller that transfers at least one pre-programmed skeletal muscle signal of said body to a plurality of pre-attached neuro-electrodes that operate all four limbs of said body to an extent that health of each limb among said four limbs allows.

18. The system of claim 10 wherein said data-processing apparatus comprises a neuro-code processing scientific computing system.

19. A method for controlling skeletal muscle, the method comprising:
selecting from a storage area, at least one skeletal muscle waveform generated in a body and carried by neurons in the body;
transmitting the at least one skeletal muscle waveform selected from the storage area to a treatment member in contact with the body; and
broadcasting the at least one skeletal muscle waveform from the treatment member to at least one skeletal muscle in the body to control the at least one skeletal muscle.

20. The method of claim 19 further comprising generating the at least one skeletal muscle waveform, wherein the at least one skeletal muscle waveform corresponds to coded waveforms generated in the body and is operative in controlling the at least one skeletal muscle.

21. The method of claim 19 further comprising recording the at least one skeletal muscle waveform in the storage area, wherein the at least one skeletal muscle waveform is generated in the body and is operative in controlling the at least one skeletal muscle.

22. The method of claim 19 generating at least one base-line skeletal muscle signal that is representative of at least one coded waveform signal generated in the body from recorded waveform signals and which exhibits a normal range of operation.

23. The method of claim 22 further comprising controlling the at least one skeletal muscle by generating a skeletal muscle signal based on comparing the at least one baseline-skeletal muscle signal with the at least one skeletal muscle waveform.
